(19) United States
(12) Patent Application Publication
(10) Pub. No.: US 2026/0200852 A1
(43) Pub. Date: Jul. 16, 2026

(54) FORMATION OF NICOTINE AND DERIVATIVES

(71) Applicant: Ready Mix Naturals, LLC, Phoenix, AZ (US)

(72) Inventors: Richard Avila, JR., Prescott Valley, AZ (US); Charlotte L. Owen, Seguin, TX (US); Geoff W. Habicht, Phoenix, AZ (US)

(73) Assignee: Ready Mix Naturals, LLC, Phoenix, AZ (US)

(21) Appl. No.: 19/445,895

(22) Filed: Jan. 12, 2026

Related U.S. Application Data
(60) Provisional application No. 63/744,075, filed on Jan. 10, 2025.

(51) Int. Cl. C07D 213/38; C07D 207/06; C07D 213/16

(52) U.S. Cl. CPC C07D 213/38; C07D 207/06; C07D 213/16

(57) ABSTRACT

A method can comprise providing a pyridine compound, providing a pyrrolidine compound, and coupling the pyridine compound and the pyrrolidine compound to form a nicotine compound.

### CROSS-REFERENCE TO RELATED APPLICATIONS

This application is a nonprovisional of, and claims priority to and the benefit of, U.S. Provisional Patent Application No. 63/744,075 , filed Jan. 10, 2025 and entitled “FORMATION OF NICOTINE AND DERIVATIVES,” which is hereby incorporated by reference herein.

### FIELD

The present disclosure generally relates to methods and processes for the production (e.g., synthetic and/or biosynthetic production) of nicotine and analogs/derivatives thereof.

### BACKGROUND

Nicotine is a naturally occurring alkaloid found in tobacco plants, which can have significant applications in the pharmaceutical, agricultural, and consumer product industries. Beyond its well-known role as a bioactive compound in tobacco products, nicotine and its analogs are being explored for their potential therapeutic applications, including treatments for neurological disorders such as Alzheimer's and Parkinson's disease. Additionally, nicotine derivatives can be utilized in developing potentially less harmful alternatives to smoking or tobacco cessation products, safer insecticides, and/or other agrochemical products.

Traditionally, nicotine is extracted from tobacco plants, a process that is labor-intensive, environmentally taxing, and subject to the limitations of agricultural productivity. Chemical synthesis of nicotine and its analogs has also been pursued. However, chemical synthesis methods often involve complex multi-step processes, use of hazardous reagents, and/or high costs, making them undesirable or unsuitable for large-scale production.

The emerging field of synthetic biology and metabolic engineering has opened new avenues for producing valuable natural products through (bio)synthetic pathways. Despite significant advances, the (bio)synthetic production of nicotine and its analogs remains challenging.

### SUMMARY

This summary is provided to introduce a selection of concepts in a simplified form. These concepts are described in further detail in the detailed description of examples of the disclosure below. This summary is not intended to necessarily identify key features or essential features of the claimed subject matter, nor is it intended to be used to limit the scope of the claimed subject matter.

In various examples, nicotine and/or a nicotine derivative (e.g., 6-methylnicotine) can be formed by a coupling reaction between a pyridine portion and a pyrrolidine portion. The pyridine portion can comprise a pyridine ring (e.g., methylated at the 2-position, and/or comprising any other suitable functional groups or moieties bonded thereto). The pyrrolidine portion can comprise a pyrrolidine ring (e.g., methylated at the nitrogen (N-methylpyrrolidine), and/or comprising any other suitable functional groups or moieties bonded thereto). Coupling of the pyridine portion and the pyrrolidine portion can take place via any suitable process, such as activating the pyridine portion (e.g., via halogenation) and reacting the pyridine portion with the pyrrolidine portion (e.g., under basic conditions).

In various examples, 6-methylnicotine (e.g., S-6-methylnicotine) can be formed by a coupling reaction between a pyridine portion (e.g., 2-methylpyridine) and a pyrrolidine portion (e.g., pyrrolidine). The pyrrolidine portion can comprise a pyrrolidine ring methylated at the nitrogen (N-methylpyrrolidine). Coupling of the pyridine portion and the pyrrolidine portion can take place via any suitable process, such as activating the pyridine portion (e.g., via halogenation) and reacting the pyridine portion with the pyrrolidine portion (e.g., under basic conditions).

A starting material for reactions and/or methods disclosed therein can comprise pyridine, or a pyridine derivative or precursor such as a pyridine derivative having a functional group (e.g., for attachment and/or reaction with a resin, enzyme, and/or the like). Another starting material can comprise pyrrolidine, or a pyrrolidine derivative or precursor. A pyrrolidine precursor can comprise a (linear) amine precursor (e.g., putrescine, ornithine, and/or an ornithine derivative). The pyrrolidine precursor can be cyclized by any suitable process to form a pyrrolidine ring moiety (e.g., the pyrrolidine portion used in the coupling reaction with the pyridine portion to form 6-methylnicotine). The pyrrolidine portion can be methylated at the nitrogen atom.

In various examples, nicotine can be formed by a coupling reaction between a pyridine portion (e.g., pyridine) and a pyrrolidine portion (e.g., pyrrolidine). The pyrrolidine portion can comprise a pyrrolidine ring methylated at the nitrogen (N-methylpyrrolidine). Coupling of the pyridine portion and the pyrrolidine portion can take place via any suitable process, such as activating the pyridine portion (e.g., via halogenation) and reacting the pyridine portion with the pyrrolidine portion.

### BRIEF DESCRIPTION OF THE DRAWINGS

A more complete understanding of exemplary embodiments of the present disclosure can be derived by referring to the detailed description and claims when considered in connection with the following illustrative figures.

FIG. 1 illustrates a schematic diagram of a nicotine compound, in accordance with various examples.

FIG. 2 illustrates a method for forming a nicotine compound, in accordance with various examples.

It will be appreciated that elements in the figures are illustrated for simplicity and clarity. For example, the details of some of the elements in the figures may be exaggerated or simplified to help to improve the understanding of illustrated embodiments of the present disclosure.

### DETAILED DESCRIPTION

The detailed description of examples are described in sufficient detail to enable those skilled in the art to practice the disclosure. It should be understood that other examples may be realized and that logical, chemical, and/or mechanical changes may be made without departing from the spirit and scope of the disclosure. Thus, the detailed description is presented for purposes of illustration only and not of limitation. For example, unless otherwise noted, the steps recited in any of the method or example descriptions may be executed in any order and are not necessarily limited to the order presented. Furthermore, a step or aspect of one method and/or example can be combined with or substituted for any step or aspect of any other method and/or example discussed herein. Any reference to singular includes plural examples, and any reference to more than one component or step may include a singular example or step. Also, any reference to attached, fixed, connected, coupled (e.g., bonded), or the like may include permanent, removable, temporary, partial, full and/or any other possible attachment option. Additionally, any reference to without contact (or similar phrases) may also include reduced contact or minimal contact.

### Definitions and Interpretations

Unless defined otherwise, all technical and scientific terms used herein have the same meaning as commonly understood by those of ordinary skill in the chemical arts and medicinal chemistry arts to which this disclosure relates. Any methods and materials similar or equivalent to those described herein can be used in the practice or testing of the subject matter of the present disclosure. As per common practice in organic chemistry, chemical structures having a chiral center either illustrated with substituents attached by wavey bonds or not illustrated three dimensionally with wedged or dashed bonds, or not labeled (R) or(S) adjacent to the chiral center or in the corresponding structural name, is assumed to represent both enantiomers (unless context indicates otherwise). Chemical structures having multiple chiral centers not presented three dimensionally or labeled as having any particular chirality are assumed to include all possible stereoisomers. Compounds of the present disclosure comprise any physiochemical or stereochemical form they may possibly assume, such as, for example, isomers, prodrugs, active metabolites, tautomers, stereoisomers, regioisomers, solvated forms, salts, and/or polymorphic forms. Amorphous forms lack a distinguishable crystal lattice and therefore lack an orderly arrangement of structural units.

As used herein, the term “alkyl,” or derivatives thereof, refers to linear or branched monovalent saturated hydrocarbon substituents, optionally substituted with one or more functional groups anywhere on or within the substituent. Unless otherwise specified, an alkyl group may contain any number of carbon atoms, such as for example, C₁-C₂₄, C₁-C₁₈, C₁-C₁₀, C₁-C₈, or C₁-C₆. Examples of alkyl substituents include, but are not limited to, methyl, ethyl, n-propyl, isopropyl, n-butyl, isobutyl, sec-butyl, tert-butyl, n-pentyl, iso-pentyl neo-pentyl, n-hexyl, iso-hexyl, octadecyl, dodecyl, and so forth (all of which being functional groups). An alkyl substituent herein may be substituted, i.e., having one or more substituent groups appended on the alkyl group or incorporated within the alkyl chain. A substitution within the alkyl substituent chain may comprise an ether, sulfide, amino, or imine linkage, i.e., —O—, —S—, —N(R′)—, or —N═, for example, or some other intervening heteroatom(s). Examples of substitution on an alkyl substituent include, but are not limited to, —CN, —N₃, —NH₂, —NHR′, —N(R′)₂, —NO₂, —NH—NH₂, —NH—NHR′, —NH—NR′₂, -halo, —SH, —SR′, —S(═O)R′, —SO₂R′, —OPO₃²⁻, —PO₃²⁻, —OH, —OR′, —C(═O)R′, —OC(═O)R′, —CO₂R′, —NHC(═O)R′, —NR′C(═O)R′, —C(═O)NHR′, —C(═O)NR′₂, alkyl, alkenyl, cycloalkyl, heterocyclyl, and aryl, wherein each R′ above is independently selected from hydrogen —H and an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅). In various examples, two R′ substituents in any of these functional groups may form a ring structure.

As used herein, the term “about” refers to a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. For example, a quantity expressed as being “about 5 wt. %” includes a variance of up to 4.5 to 5.5 wt. %.

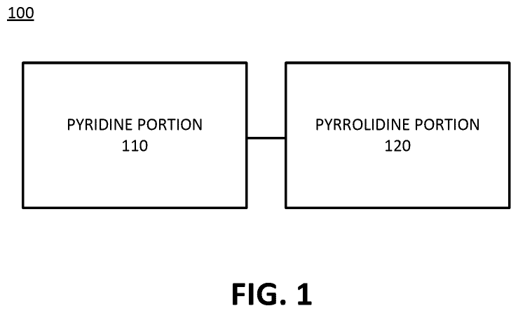

With reference to FIG. 1, nicotine and nicotine derivatives (such as nicotine compound 100 shown in FIG. 1) can comprise a pyridine portion 110 and a pyrrolidine portion 120. Nicotine compound 100 can comprise nicotine or any nicotine derivative. Pyridine portion 110 can comprise a pyridine ring. The pyridine ring can comprise any suitable functional groups and/or moieties bonded thereto at any suitable or desired position. Pyrrolidine portion 120 can comprise a pyrrolidine ring. The pyrrolidine ring can comprise any suitable functional groups and/or moieties bonded thereto at any suitable or desired position.

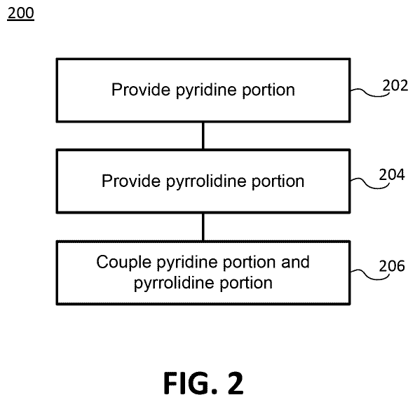

Nicotine and nicotine derivatives can be formed by any suitable method. For example, with additional reference to FIG. 2, method 200 can comprise providing a pyridine portion 110 (step 202), providing a pyrrolidine portion 120 (step 204), and/or coupling pyridine portion 110 and pyrrolidine portion 120 to form nicotine or the desired nicotine derivative. Providing the pyridine portion (step 202) can comprise forming a pyridine portion (e.g., from a pyridine precursor), providing a pyridine ring (e.g., with or without any desired functional groups and/or moieties coupled thereto), and/or coupling desired functional groups and/or moieties to a pyridine precursor and/or pyridine ring, as discussed in the disclosure and examples herein. Providing the pyrrolidine portion (step 204) can comprise forming a pyrrolidine portion (e.g., from a pyrrolidine precursor), providing a pyrrolidine ring (e.g., with or without any desired functional groups and/or moieties coupled thereto), and/or coupling desired functional groups and/or moieties to a pyrrolidine precursor and/or pyrrolidine ring, as discussed in the disclosure and examples herein. Coupling the pyridine portion and the pyrrolidine portion (step 206) can comprise any suitable coupling method and/or mechanism, such as those discussed herein.

### Nicotine Derivatives

Nicotine derivatives (i.e., analogs) can comprise the chemical structure of nicotine and include additional functional groups, moieties, and/or other atoms or structures bonded/coupled thereto. A derivative of nicotine can comprise methylnicotine (e.g., 6-methylnicotine). There can be various methods of forming (e.g., synthesizing) nicotine derivatives, for example, methylnicotine, more specifically, 6-methylnicotine. A nicotine derivative can have a pyridine portion comprising a pyridine ring with any respective functional groups and/or moieties coupled thereto, and a pyrrolidine portion with any respective functional groups and/or moieties coupled thereto. For example, 6-methylnicotine can have a pyridine portion comprising a pyridine ring with a methyl group at the 6-position, and a pyrrolidine portion. The pyrrolidine portion can have a methyl group at the nitrogen atom (i.e., at the 1-position). Formation of a certain enantiomer of 6-methylnicotine can be desired, such as S- 6-methylnicotine. Thus, reference herein to forming 6-methylnicotine can also refer to forming a desired enantiomer (e.g., S-6-methylnicotine), and/or reference herein to forming a certain enantiomer (e.g., S-6-methylnicotine) can also refer to forming the general compound (6-methylnicotine), without preference or reference to a specific enantiomer.

Starting compounds for (stereocontrolled) synthesis of a nicotine derivative (e.g., 6-methylnicotine) can comprise pyridine or a pyridine derivative or precursor, and pyrrolidine or a pyrrolidine derivative or precursor. A pyrrolidine precursor can comprise a linear amine (e.g., putrescine, ornithine, and/or an ornithine derivative).

“Pyridine,” “pyridine portion,” “pyridine compound,” “pyridine intermediate,” and/or other like terms used herein regarding a reaction(s) to form 6-methylnicotine can comprise pyridine, a pyridine derivative or precursor, and/or 2-methylpyridine. “Pyridine,” “pyridine portion,” “pyridine compound,” “pyridine intermediate,” and/or other like terms used herein (which can be used interchangeably) regarding a compound used in a reaction to form nicotine or a nicotine derivative (e.g., 6-methylnicotine) can comprise pyridine, a pyridine derivative or moiety, a pyridine precursor, a pyridine intermediate, and/or a compound comprising a pyridine ring. In various examples, for 6-methylnicotine formation, a methyl group can be regioselectively added to a pyridine ring (e.g., a pyridine molecule, compound, portion, or moiety) at the 2-position. For formation of another nicotine derivative, a respective functional group or moiety can be added to the pyridine portion at a desired position.

A pyrrolidine precursor can be cyclized into pyrrolidine. Even if not mentioned in a specific example discussed herein, the pyrrolidine ring can be regioselectively methylated at the nitrogen by any suitable process. “Pyrrolidine,” “pyrrolidine portion,” “pyrrolidine compound,” “pyrrolidine intermediate,” and/or other like terms used herein regarding a reaction(s) to form 6-methylnicotine can comprise methylpyrrolidine (pyrrolidine methylated at the nitrogen atom). “Pyrrolidine,” “pyrrolidine portion,” “pyrrolidine compound,” “pyrrolidine intermediate,” and/or other like terms used herein (which can be used interchangeably) regarding a compound used in the reaction to form nicotine or a nicotine derivative (e.g., 6-methylnicotine) can comprise pyrrolidine, a pyrrolidine derivative or moiety, a pyrrolidine precursor, a pyrrolidine intermediate, and/or a compound comprising a pyrrolidine ring. In various examples, for 6-methylnicotine formation, a methyl group can be regioselectively added to the pyrrolidine ring (e.g., a pyrrolidine molecule, compound, portion, or moiety) at the nitrogen. For formation of another nicotine derivative, a respective functional group or moiety can be added to the pyrrolidine portion at a desired position.

The pyridine portion (e.g., 2-methylpyridine) and pyrrolidine portion (e.g., a methylated pyrrolidine) can be coupled such that the pyrrolidine portion couples to the pyridine portion at the desired position (e.g., the 5-position or 3-position) of the pyridine ring. In various examples, the pyridine portion can be activated by halogenation (e.g., using N-bromosuccinimide (NBS) or iodine) to form an electrophilic derivative (e.g., a halogen atom can be bonded to the pyridine ring at the 5-position or 3-position). The activated pyridine portion can be reacted with the pyrrolidine portion in the presence of a strong base (e.g., K₂CO₃ or NaH) to couple the pyridine portion and pyrrolidine portion to form (preferentially) the desired nicotine compound, e.g., 6-methylnicotine (e.g., a majority, or more than 60%, 70%, 80%, or 90%, of the 6-mehtylnicotine formed can be the S-enantiomer rather than the R-enantiomer).

The described reaction, for example, can be a nucleophilic aromatic substitution reaction (SNAr) to form a C—C bond between the 3 or 5-position of the activated pyridine ring and the 2-position of the pyrrolidine ring. For activation of the pyridine portion, 2-methylpyridine can undergo electrophilic aromatic substitution with a halogenating agent like NBS (providing Br⁺) or iodine. The electrophile attacks the 3 or 5-position of the pyridine ring (e.g., directed by the activating methyl group at position 2 and the inherent reactivity of the pyridine system). Loss of a proton from the sigma complex restores aromaticity, yielding a 5-halo-2-methylpyridine (e.g., 5-bromo-2-methylpyridine). Subsequently, deprotonation can occur, wherein a strong base (e.g., NaH or K₂CO₃) deprotonates an alpha C—H bond (at the 2-position) of the methylated pyrrolidine (1-methylpyrrolidine). This generates a nucleophilic carbanion at the 2-position of the pyrrolidine ring. Nucleophilic addition can occur, wherein the pyrrolidine carbanion attacks the carbon at the 5-position of the halogenated pyridine, adding to the aromatic ring and forming a negatively charged intermediate. The negative charge is delocalized across the pyridine ring, with partial stabilization by the nitrogen atom. Then, the leaving group can be eliminated, wherein the halide ion (e.g., Br⁻) departs from the sp³-hybridized carbon, restoring the aromaticity of the pyridine ring and forming the C—C coupled product, 6-methylnicotine.

The pyridine portion and the pyrrolidine portion can be coupled via other suitable methods utilizing catalysts, enzymes, and/or any other suitable compounds or materials, discussed herein.

The desired product can be extracted (e.g., by a liquid-liquid extraction using ethyl acetate and/or dichloromethane (DCM)) to isolate the crude product comprising 6-methylnicotine (or other nicotine compound). The product can be purified by any suitable method, such as silica gel chromatography, column chromatography, high performance chromatography, recrystallization, and/or the like. Resulting 6-methylnicotine (or other nicotine compound) can be crystallized for stability and high purity. The extracted and/or purified product, if a nicotine derivative (e.g., 6-mehtylnicotine), can be free and devoid of nicotine.

To verify that the product comprises the desired 6-methylnicotine (or other nicotine compound), product verification can be conducted through any suitable method. For example, nuclear magnetic resonance (NMR) spectroscopy (e.g., 1H, 13C), Fourier transform infrared (FTIR) spectroscopy, gas chromatography, and/or mass spectrometry can be utilized for structure elucidation. To determine the enantiomeric purity (e.g., enantiomeric excess) of the desired 6-methylnicotine (or other nicotine compound) (e.g., the S-enantiomer), the product can be analyzed using chiral column high-performance liquid chromatography (HPLC), polarimetry, or any other suitable method.

The following examples recite a series of steps for the formation (e.g., synthesis), extraction, and/or purification of nicotine or nicotine derivatives (e.g., 6-methylnicotine). It should be understood that the steps from various examples can be combined and/or performed in any suitable combination or order. For example, one or more steps from one example can be implemented with, or substituted for, one or more steps from another example, as desired and/or appropriate.

### Example 1

In various examples, 6-methylnicotine can be formed via catalytic hydrogenation and/or regioselective alkylation. This method uses catalytic reduction and selective alkylation techniques to synthesize 6-methylnicotine.

A starting material can comprise a pyridine compound (e.g., pyridine). The pyridine compound can be selectively reduced using a palladium catalyst, such as Pd/C or Pd(OAc)₂. The pyridine compound can be reacted with the palladium catalyst under hydrogen gas (H₂) at controlled pressure (e.g., 1-5 atm) and temperature (40-60° C., or about 50° C.). A product of this reaction can be a tetrahydropyridine intermediate. Catalysts comprising rhodium and/or alternative/additional reductants such as NaBH4 can be used.

The tetrahydropyridine intermediate can be regioselectively alkylated to introduce a methyl group at the 2-position of the pyridine ring portion. For example, the tetrahydropyridine intermediate can be reacted with a methylating agent (e.g., methyl iodide or dimethyl sulfate). Such a reaction can take place under basic conditions (e.g., the presence of a base such as NaHCO₃, K₂CO₃, lithium diisopropylamide (LDA), and/or NaH). A solvent for the methylation reaction can comprise dimethylformamide (DMF) and/or acetonitrile. Temperature for the reaction can be 60-100° C, or about 80° C. This methylation reaction can result in a product comprising an alkylated tetrahydropyridine, which comprises a methyl group at the 2-position of the pyridine portion.

The alkylated tetrahydropyridine can be oxidized to 2-methylpyridine using an oxidant, such as MnO₂ and/or 2,3-Dichloro-5,6-dicyano-1,4-benzoquinone (DDQ).

Another starting material can comprise a pyrrolidine precursor, such as a (linear) amine precursor (e.g., putrescine and/or an ornithine derivative, such as L-Ornithine), an imine-based precursor (e.g., butyrolactam and/or Schiff base of 4-aminobutanal), and/or an aldehyde-amine precursor (e.g., 4-aminobutanal and/or 4-aminobutyric acid). The pyrrolidine precursor can be cyclized in mildly acidic (e.g., pH of 4-6.9, 4-6, 5-6.9, or 5.5-6.5) and/or mild thermal (e.g., temperatures sufficient to induce cyclization without degrading sensitive intermediates, such as 50-100° C.) conditions to form a pyrrolidine portion (e.g., comprising a pyrrolidine ring). A catalyst, such as acetic acid and/or hydrochloric acid, can be used at low concentrations (e.g., 0.1-1 M). A solvent can comprise aqueous and/or alcohol-based (e.g., ethanol and/or methanol) solvents. For higher temperature reactions, for example at 120-180° C. (e.g., for reactions without acidity), a solvent can comprise toluene and/or THF.

The pyridine portion and the pyrrolidine portion can be coupled to form the desired 6-methylnicotine. The pyridine portion can be activated by halogenation (e.g., using N-bromosuccinimide (NBS) and/or iodine) to form an electrophilic derivative thereof. The activated pyridine portion can be reacted with the pyrrolidine portion in the presence of a strong base (e.g., K₂CO₃ or NaH) to couple the pyridine portion and pyrrolidine portion, forming 6-methylnicotine. Solvent for this coupling reaction can comprise a polar aprotic solvent (e.g., acetonitrile or DMF), which can enhance nucleophile reactivity without protonating the carbanion. For example, halogenation can occurs at the 5-position of the pyridine portion. The 2-methyl group can direct ortho/para in electrophilic aromatic substitution, favoring the 5-position in pyridine due to inherent reactivity patterns. NBS (radical bromination conditions often used, e.g., with AIBN initiator in CCl₄) or iodine can yield a 5-halo-2-methylpyridine (typically 5-bromo as Br can be a leaving group). A (strong) base (NaH or K₂CO₃) can generate an alpha-carbanion at the 2-position of the pyrrolidine ring (adjacent to nitrogen). The pyrrolidine carbanion can act as a carbon nucleophile, adding to the electron-deficient carbon at the 5-position of the halogenated pyridine (activated by the ring nitrogen, similar to nitro-group activation in classic SNAr). Elimination of the halide (Br⁻ or I⁻) restores aromaticity, yielding the coupled product.

The product (e.g., a solution) comprising 6-methylnicotine can be extracted via any suitable method. For example, a liquid-liquid extraction (e.g., a two-phase extraction) can be performed using ethyl acetate and/or dichloromethane (DCM) to isolate the crude product comprising 6-methylnicotine. The product comprising 6-methylnicotine can be purified (e.g., via a silica gel chromatography process and/or a recrystallization process from ethanol or methanol). The 6-methylnicotine can be crystallized to facilitate/maintain stability and/or high purity. Crystallization can occur by converting the 6-methylnicotine to a (solid) salt and/or cocrystal form (e.g., salt formation with acids like salicylic acid, malic acid, or tartaric acid).

To verify formation of 6-methylnicotine, NMR spectroscopy, FTIR spectroscopy, gas chromatography, and/or mass spectrometry can be performed. To determine enantiomeric purity, chiral HPLC and/or polarimetry can be performed.

Formation of 6-methylnicotine via catalytic hydrogenation and/or regioselective alkylation can have various benefits including regioselectivity (controlled reaction conditions can allow selective functionalization of the pyridine ring), stereoselectivity (chiral catalysts can facilitate high enantiomeric excess), scalability (can be suitable for both lab-scale and industrial-scale synthesis), and/or cost-effectiveness (can utilize readily available reagents and catalysts). The steps and methods in this example can be used for synthesizing 6-Methylnicotine for pharmaceutical and industrial applications due to its simplicity, scalability, and compatibility with green chemistry principles¹ (e.g., atom economy, catalysis, energy efficiency, safer solvents, minimized hazardous byproducts, and/or renewable feedstocks).

### Example 2

In various examples, 6-methylnicotine can be formed via electrophilic aromatic substitution (EAS) and/or asymmetric catalysis. EAS can selectively introduce a methyl group at the 2-position of a pyridine portion (e.g., pyridine). Asymmetric catalysis can construct a pyrrolidine ring portion. This example leverages the regioselectivity of electrophilic substitution reactions for functionalizing the pyridine portion and uses chiral catalysts to construct the pyrrolidine portion.

A starting material can comprise a pyridine portion (e.g., pyridine). The pyridine portion can be selectively halogenated (e.g., brominated) at the 2-position. For example, halogenation can occur using N-bromosuccinimide (NBS) in the presence of a Lewis acid (e.g., AlCl₃) as a catalyst. As another example, halogenation can occur via halogenation of pyridine N-oxide (e.g., with POBr₃ or Ac₂O/Br₂). As another example, halogenation can occur via deprotonation at the 2-position (C2) with strong bases (e.g., n-BuLi or LDA, which can include additives like TMEDA), followed by quenching with Br₂ or C₂Br₆. As another example, halogenation can occur via phosphine-mediated activation or Zincke salt intermediates for ¹“Green chemistry” as defined in Anastas, et al., Green Chemistry: Principles and Practice,” CENTER FOR GREEN CHEMISTRY AND GREEN ENGINEERING AT YALE, published Nov. 20, 2009, which is hereby incorporated by reference herein. regioselective halogenation (e.g., at C3 or tunable positions). Methylation can occur by substituting the halogen (e.g., bromine) on the pyridine portion with a methyl group (e.g., via a palladium-catalyzed cross-coupling reaction (e.g., using methylboronic acid in a Suzuki-Miyaura coupling)). A catalyst can comprise Pd(PPh₃)₄ and/or similar palladium complexes. A solvent for such a reaction can comprise DMF and/or toluene.

To form a pyrrolidine portion, a second starting material can comprise a pyrrolidine precursor, such as ornithine or an amine precursor. The pyrrolidine precursor can be cyclized using a chiral transition metal catalyst, such as 2,2′-bis(diphenylphosphino)-1,1′-binaphthyl (BINAP, e.g., Rh-BINAP) and/or 1,2-Diphenyl-1,2-ethylenediamine (DPEN), and/or a N-tosylated derivative thereof (TsDPEN, e.g., Ru-TsDPEN), to form the pyrrolidine portion. A methyl group can be introduced on the nitrogen atom of the pyrrolidine portion using formaldehyde (e.g., in the presence of a chiral auxiliary or catalyst). As another example, a pyrrolidine precursor comprising L-Ornithine (chiral, natural precursor) can decarboxylate to putrescine (1,4-diaminobutane), which can be mono-N-methylated early to N-methylputrescine. Oxidative deamination (biomimetic or chemical) can yield 4-(methylamino)butanal, which can (spontaneously) cyclize to the N-methyl-Δ¹-pyrrolinium cation (a reactive imine). Pyrrolidine formation can take place in aqueous ethanol or methanol (e.g., using formic acid/triethylamine azeotrope or H₂/isopropanol as H-source). Reactions can occur at room temperature (25° C.) to 40-60° C.

The pyridine portion and the pyrrolidine portion can be coupled to form the desired 6-methylnicotine. The pyridine portion can be converted into an electrophilic derivative (e.g., activated), for example, by halogenation using, e.g., NBS, bromine, and/or iodine. The activated pyridine portion can be reacted with the pyrrolidine portion in the presence of a strong base (e.g., K₂CO₃ or NaH) to couple the pyridine portion and the pyrrolidine portion, forming 6-methylnicotine. Solvent for this coupling reaction can comprise a polar aprotic solvent (e.g., acetonitrile or DMF). The temperature and stoichiometry of this reaction can be controlled to maximize yield and avoid undesired byproducts.

The product (e.g., a solution) comprising 6-methylnicotine can be purified and isolated. For example, the product comprising 6-methylnicotine can be extracted (e.g., via liquid-liquid extraction using ethyl acetate and/or DCM). The product comprising 6-methylnicotine can be purified via a column chromatography process and/or a recrystallization process from ethanol or methanol. The 6-methylnicotine can be crystallized to facilitate/maintain stability and/or high purity.

To verify formation of 6-methylnicotine, NMR spectroscopy, FTIR spectroscopy, gas chromatography, and/or mass spectrometry can be performed. To determine enantiomeric purity, chiral HPLC and/or polarimetry can be performed.

Formation of 6-methylnicotine via EAS and asymmetric catalysis can have various benefits including regioselectivity (EAS can facilitate precise functionalization of the pyridine ring), stereoselectivity (asymmetric catalysis can yield high enantiomeric excess of the S-enantiomer), versatility (can be compatible with a variety of substrates and reaction conditions), and/or scalability (EAS and asymmetric catalysis can be scaled methods in organic synthesis). The steps and methods in this example can be used for synthesizing 6-methylnicotine at both small and industrial scales, particularly when high regio- and stereoselectivity are desired, such as in pharmaceutical and fine chemical industries.

### Example 3

In various examples, 6-methylnicotine can be formed via radical-based synthesis, which can utilize controlled radical reactions to construct the 6-methylnicotine molecule. Radical chemistry can provide unique reactivity for selective functionalization and stereocontrol in complex molecules.

A starting material can comprise a pyridine portion (e.g., pyridine). Radical methylation of the pyridine portion can comprise generating a methyl radical from a methylating agent (e.g., trimethylsilylmethyl chloride, acetone, diacetyl peroxide, tert-butyl hydroperoxide (TBHP), dimethyl sulfoxide (DMSO), acetic acid/peroxydisulfate, and/or methanol), for example, using a photocatalyst (e.g., [Ru(bpy)₃]Cl₂) and/or an organic photocatalyst (e.g., Eosin Y)) and/or a light source (e.g., (blue) LEDs). The methyl radical can be directed to the 2-position of the pyridine portion using electron-withdrawing directing groups or solvent effects. Radical methylation, or any steps thereof, can take place in acetonitrile and/or DMF. For example, selective C—H functionalization of heteroarenes can occur. The light intensity, temperature (e.g., about 25° C.), and/or reaction time can be controlled/optimized to maximize regioselectivity.

To form a pyrrolidine portion, a second starting material can comprise a pyrrolidine precursor, such as ornithine or an amine precursor. Radical cyclization to form the pyrrolidine portion can comprise using a peroxide initiator (e.g., di-tert-butyl peroxide) or photocatalytic conditions to generate a nitrogen-centered radical. Peroxide initiators such as di-tert-butyl peroxide (DTBP) can decompose thermally (e.g., >100°C) to tert-butoxy radicals, which abstract H or initiate homolysis of N—X bonds (X=halogen, O, etc.), yielding amidyl, aminyl, or sulfonamidyl radicals. A pyrrolidine ring can be formed via intramolecular radical addition. This process can comprise generating a nitrogen-centered radical (NCR) from a pyrrolidine precursor (e.g., derived from L-ornithine→putrescine→N-methylputrescine or similar linear amine), followed by intramolecular radical addition to form the pyrrolidine ring. Photocatalytic conditions (e.g., visible light with Ir/Ru dyes, organic photocatalysts like Eosin Y, or metal-free systems) can enable milder single electron transfer (SET) oxidation/reduction to generate NCRs from precursors like N-chloramines, hydrazones, oximes, or sulfonamides. In various examples, cyclization can include an NCR undergoing 5-exo-trig cyclization onto a pendant unsaturated bond (e.g., alkene, alkyne, or activated C═C in the chain), forming a new C—N bond and a carbon radical intermediate. The intermediate can be trapped (e.g., by H-abstraction, halogen transfer, or further cyclization), yielding the pyrrolidine. In various examples involving ornithine and/or amine precursors, cyclization can include conversion to a linear chain with N-halogenation (e.g., N-chloro or N-iodo derivative) or other NCR precursor (e.g., sulfonamide with alkene tether). In various examples involving protonated N-haloamines (e.g., N-bromo or N-chloro secondary amines), cyclization can include forming a carbon radical that captures halogen, followed by base-induced cyclization to the pyrrolidine (conditions for which can include acid, heat, and/or ultraviolet light).

The pyridine portion and the pyrrolidine portion can be coupled to form the desired 6-methylnicotine. The pyridine portion can be activated by using a halogenation agent (e.g., NBS, bromine, and/or iodine) to prepare the pyridine portion for nucleophilic substitution. The activated pyridine portion can be reacted with the pyrrolidine portion under radical conditions using a photocatalyst or radical initiator (e.g., using visible-light irradiation in the presence of a base (e.g., potassium carbonate)) to couple the pyridine portion and the pyrrolidine portion, forming 6-methylnicotine. Solvent for this coupling reaction can comprise a polar aprotic solvent (e.g., acetonitrile or DMF). The temperature and stoichiometry of this reaction can be controlled to maximize yield and avoid undesired byproducts.

The product (e.g., a solution) comprising 6-methylnicotine can be purified and isolated. Radicals can be quenched using antioxidants (e.g., ascorbic acid) to stabilize the reaction mixture. The product comprising 6-methylnicotine can be extracted (e.g., via liquid-liquid extraction using ethyl acetate and/or DCM). The product comprising 6-methylnicotine can be purified via a silica gel chromatography process or a recrystallization process.

To verify formation of 6-methylnicotine, NMR spectroscopy, FTIR spectroscopy, gas chromatography, and/or mass spectrometry can be performed. To determine enantiomeric purity (e.g., enantiomeric excess), chiral HPLC and/or optical rotation can be performed.

Formation of 6-methylnicotine via radical-based synthesis can have various benefits including unique reactivity (radical reactions can access functionalization patterns difficult to achieve with traditional methods), regioselectivity (radicals can selectively react at electronically or sterically favored positions), mild conditions (photocatalytic reactions can occur at about room temperature under about atmospheric pressure), and/or scalability (radicals can be easily generated in continuous flow systems, making this approach scalable). Radical-based synthesis can be advantageous for synthesizing complex molecules with challenging substitution patterns, such as 6-methylnicotine, and can be suitable for both pharmaceutical and fine chemical industries.

### Example 4

In various examples, 6-methylnicotine can be formed via biomimetic synthesis, which can be similar to the natural biosynthetic pathways of nicotine-related alkaloids in plants. Biomimetic synthesis can be similar to the enzymatic steps of natural pathways, and can substitute with small molecule catalysts or mild chemical conditions.

In various examples, the pyridine ring portion of 6-methylnicotine can be synthesized by reacting alpha-iminosuccinic acid with 2,3-dihydroxybutyraldehyde-3-phosphate in the presence of the enzyme quinolinic acid synthase (or quinolinate synthase) (QS) to produce 6-methylquinolinic acid. Alpha-iminosuccinic acid can be formed from L-aspartate oxidation by L-aspartate oxidase (NadB). Note that in the tobacco plant, alpha-iminosuccinic acid can undergo QS-catalyzed cyclization with dihydroxyacetone phosphate (DHAP) to produce quinolinic acid. Quinolinate synthase (QS, or NadA) can catalyze the complex condensation of iminoaspartate with DHAP, involving dephosphorylation, isomerization, cyclization, and/or dehydrations to form quinolinic acid (QA). In various examples, alpha-iminosuccinic acid can undergo QS-catalyzed cyclization with glyceraldehyde-3-phosphate (gly-3-P) to produce quinolinic acid. Replacing gly-3-P (3 carbons) with 2,3-dihydroxybutyraldehyde-3-phosphate (4 carbons) can provide the methyl group that can end up in the 6-position of quinolinic acid in this enzyme-catalyzed regiospecific cyclization.

In various examples, the resulting 6-methylquinolinic acid from this biomimetic reaction can be possibly converted directly to 6-methylnicotine in a one-pot reaction with the reactive pyrrolidine portion of the molecule (e.g. the N-methylpyrrolidinium cation) using the enzymes present in the biosynthesis pathway.

To prepare a pyridine portion, a starting material can comprise a precursor in natural nicotine biosynthesis (e.g., nicotinic acid). The carboxylic acid group can be removed from nicotinic acid by using a decarboxylation reagent (e.g., copper(II) oxide and/or an iron salt), yielding pyridine. In various examples, nicotinic acid (niacin) can be mixed with copper carbonate or oxide, and heated about 220-250° C., and distill pyridine (e.g., as an azeotrope with water, purified by base treatment and redistillation). Such a reaction may occur in an inert atmosphere and/or comprising iron salts. Methylation of the 2-position of the pyridine portion can comprise using dimethyl carbonate or methyl iodide in the presence of a Lewis acid (e.g., AlCl₃ or BF₃), which can be similar to the role of methyltransferases.

To form a pyrrolidine portion, a second starting material can comprise a pyrrolidine precursor, such as a natural amino acid precursor for pyrrolidine rings (e.g., ornithine). Ornithine can be cyclized to form the pyrrolidine portion using mild acidic (e.g., pH of 4-6.9, 4-6, 5-6.9, or 5.5-6.5) or mild basic (e.g., pH of 7.1-10, 8-10, or 7.5-8.5) conditions, similar to the natural enzymatic reaction catalyzed by ornithine decarboxylase. Ornithine decarboxylase (ODC) can catalyze the decarboxylation of L-ornithine to putrescine (1,4-diaminobutane) (e.g., ornithine forms a Schiff base with pyridoxal phosphate (PLP), decarboxylates to a quinoid intermediate, and rearranges to release putrescine (with retention of configuration at the α-carbon in some systems). In various examples, arginine decarboxylase (ADC) on arginine→agmatine→N-carbamoylputrescine→putrescine. A methyl group can be introduced on the nitrogen atom of the pyrrolidine portion using a biomimetic approach with formaldehyde and a chiral catalyst (e.g., a BINOL-based Lewis acid). For example, putrescine N-methyltransferase (PMT) can methylate putrescine to N-methylputrescine. N-Methylputrescine oxidase (MPO) can oxidatively deaminate N-methylputrescine to 4-(methylamino)butanal. This aldehyde, 4-(methylamino)butanal, can cyclize (e.g., spontaneously) to the N-methyl-Δ¹-pyrrolinium cation, the reactive intermediate incorporating into nicotine (condensing with a pyridine-derived precursor via enzymes like A622 and berberine bridge enzyme-like proteins).

The pyridine portion and the pyrrolidine portion can be coupled to form the desired 6-methylnicotine. The pyridine portion can be activated by using a mild halogenation agent (e.g., NBS) to create an electrophilic site on the pyridine ring portion. To achieve nucleophilic substitution, the activated pyridine portion can be reacted with pyrrolidine portion in the presence of a mild base (e.g., K₂CO₃) in an organic solvent such as acetonitrile or dimethylsulfoxide (DMSO).

The product (e.g., a solution) comprising 6-methylnicotine can be purified and isolated. The product comprising 6-methylnicotine can be extracted (e.g., via liquid-liquid extraction using ethyl acetate and/or DCM). The product comprising 6-methylnicotine can be purified via a column chromatography process or a recrystallization process. The 6-methylnicotine can be isolated as a pure crystalline compound.

To verify formation of 6-methylnicotine, NMR spectroscopy, FTIR spectroscopy, gas chromatography, and/or mass spectrometry can be performed to confirm molecular structure. To determine enantiomeric purity (e.g., enantiomeric excess), chiral HPLC and/or optical rotation can be performed.

Formation of 6-methylnicotine via biomimetic synthesis can have various benefits including being environmentally friendly (can operate under mild conditions, such as room temperature and atmospheric pressure, avoiding harsh reagents or extreme temperatures), inspired by nature (follows and/or mimics mechanisms effective in natural biosynthesis), regioselectivity and stereoselectivity (can simplify the control of selectivity by mimicking enzymatic pathways), and/or scalability (adaptable for both lab-scale and industrial-scale synthesis). Biomimetic synthesis can be advantageous for producing nicotine analogs and related alkaloids with high specificity and can be suited for small-scale, high-purity applications such as pharmaceuticals or research compounds.

### Example 5

In various examples, 6-methylnicotine can be formed via template-directed synthesis, which can leverage pre-organized molecular scaffolds or templates to guide the regio- and stereoselective assembly of 6-methylnicotine. This strategy can be useful for complex molecules requiring precise spatial arrangement. A molecular template can organize reactants spatially, ensuring regioselectivity. The template can provide steric and electronic environments to induce chirality in the reaction products.

To prepare a template, a polyfunctional molecule (e.g., a macrocycle or multidentate ligand) with binding sites for both pyridine and pyrrolidine precursors can be used (e.g., a chiral macrocyclic structure such as a crown ether or cyclophane). The template can be modified with reactive groups to anchor intermediates via hydrogen bonding or covalent interactions. For example, a custom chiral cyclophane or pyridine-binding crown ether (e.g., 18-crown-6 with chiral binaphthyl arms or amide H-bond arrays for dual pyridine N-coordination and pyrrolidine NH/CH binding) can be specified. Zn-porphyrin and/or urea pockets can be added for selectivity.

In the assembly (i.e., preparation) of the pyridine portion (e.g., 2-methylpyridine), a pyridine ring portion (a starting material) can be introduced to the template. The pyridine ring portion can be anchored to the template via coordination (e.g., N-coordination) or hydrogen bonding. In various examples, the nitrogen in the pyridine can H-bond to the template amides and/or coordinates to Lewis acidic sites (e.g., Cu²⁺ in crown cavity), e.g., in CHCl₃/MeOH. In various examples, (temporary) imine/oxime formation from 2-pyridinecarboxaldehyde precursor can occur.

Methylation of the 2-position of the pyridine ring portion can comprise adding a methyl donor (e.g., methyl iodide or dimethyl sulfate) in the presence of a base and a directing group provided by the template. The template can restrict methylation to the 2-position of the pyridine ring portion. For example, a template-attached group can act as an ortho-director, favoring deprotonation (with base) or electrophilic attack at the 2-position. Steric bulk from the macrocycle can block other positions on the pyridine ring portion. The methyl donor can then selectively bond to the pyridine ring portion. In various examples, the template can position a carbamate/OMOM (methoxymethyl ether) direct metalation group ortho to the pyridine nitrogen. S-BuLi/TMEDA (−78° C., THF) can be added, which can lithiate C2 selectively. The reaction can be quenched with MeI. Such reaction(s) can occur under inert conditions (e.g., N₂), at about −78° C. to room temperature for any suitable time (e.g., 1-4 hours).

To form a pyrrolidine portion, a pyrrolidine precursor (e.g., an amine and/or aldehyde) can be introduced to the template. In various examples, protonated N-methylputrescine and/or (S)-1-methylpyrrolidine-2-carbaldehyde can binds via 3-5 H-bonds to chiral urea/amide sites and/or cation-π in cyclophane cavity. In various examples, a Schiff base can bind with template aldehyde, preserving S-chirality from L-ornithine precursor. A reducing agent (e.g., NaBH₄ or H₂ gas with a catalyst) can be used to cyclize the precursor into the pyrrolidine portion.

The pyridine portion and the pyrrolidine portion can be coupled to form the desired 6-methylnicotine. The pyridine portion and the pyrrolidine portion can be positioned in proximity on the template for the coupling reaction. For example, one site (e.g., amide/urea H-bond arrays or cation-π pockets) can anchor the pyridine via N-coordination or π-stacking; another (e.g., protonatable groups or secondary ammonium recognition) can bind the pyrrolidine precursor (e.g., protonated N-methylputrescine or aldehyde derivative). The template's rigid structure can restrict rotational freedom, aligning reactive sites (e.g., <5-10 Å apart) and biasing transition states for regio- and stereoselectivity. A nucleophilic substitution reaction (e.g., SN2) can be used to attach pyrrolidine to the pyridine portion. This reaction can be performed under mild conditions (e.g., room temperature with a base such as K₂CO₃). In various examples, the coupling reaction can comprise halogenation at the 5-poisition of the pyridine portion (NBS, CCl₄), deprotonate pyrrolidine at 2-position (e.g., via strong base such as NaH), and SNAr can occur in the template cavity (e.g., under DMF, 50-80° C.).

The product comprising 6-methylnicotine can be released and/or disassociated from the template. Mild acidic (e.g., pH of 4-6.9, 4-6, 5-6.9, or 5.5-6.5) or mild basic (e.g., pH of 7.1-10, 8-10, or 7.5-8.5) conditions can be used to release 6-methylnicotine from the template without, or mitigating the risk of, damaging the product. Dissociation can occur by adding competing solvents (e.g., polar protic like water/MeOH) and/or ligands, and/or by pH adjustment (e.g., mild base like NaHCO₃ to disrupt H-bonds). Perform at room temperature (RT) under inert atmosphere (N₂) to avoid oxidation; yields >95% recovery with no degradation. Covalent tethers (e.g., imine, boronate, or disulfide links) can be selectively cleaved (e.g., imine/oxime can be hydrolyzed with dilute acid (0.1-1 M HCl or acetic acid in MeOH/H₂O, pH 3-5, RT, 1-2 h); boronate ester can be cleaved by adding pinacol or water (e.g., at about room temperature, about neutral pH); disulfide can be cleaved by reducing with Zn dust/AcOH or dithiothreitol (DTT) (e.g., at about neutral pH, about room temperature)). The 6-methylnicotine can be released from the template without (significantly) altering the structure of the template. Thus, the template can be regenerated and recycled for subsequent reactions. The template, after the 6-methylnicotine is extracted, can be isolated by evaporation, precipitation (e.g., in hexane), and/or chromatography.

The product (e.g., a solution) comprising 6-methylnicotine can be purified and isolated. The product comprising 6-methylnicotine can be extracted (e.g., using an organic solvent, such as DCM). The product comprising 6-methylnicotine can be purified via a column chromatography process or a recrystallization process.

To verify formation of 6-methylnicotine, NMR spectroscopy, gas chromatography, and/or mass spectrometry can be performed to confirm molecular structure. To determine enantiomeric purity (e.g., enantiomeric excess), chiral HPLC and/or polarimetry can be performed.

Formation of 6-methylnicotine via template-directed synthesis can have various benefits including being high precision (can facilitate regio- and stereoselectivity through spatial control), reusability (templates can be reusable, reducing material costs), reduced byproducts (can minimize side reactions by organizing reactants in a controlled environment), and/or versatility (the template can be adapted for other related alkaloids or derivatives). Template-directed synthesis can be particularly useful for small-scale, high-purity applications such as pharmaceutical research or the production of complex natural product derivatives.

### Example 6

In various examples, 6-methylnicotine can be formed via flow chemistry synthesis, which integrates continuous reaction systems to synthesize 6-methylnicotine. This method can facilitate precise control of reaction conditions, scalability, and high efficiency. Flow chemistry can involve conducting chemical reactions in continuous flow systems rather than traditional batch reactors. Reactants can flow through microreactors or tubular systems where reactions occur under well-controlled conditions.

A starting material can comprise a pyridine portion. Methylation of the pyridine portion can comprise introducing the pyridine portion and a methylating agent (e.g., methyl iodide or dimethyl carbonate) into a flow reactor. A palladium-based catalyst (e.g., Pd/C) packed in the flow reactor can be used to promote selective methylation at the 2-position of the pyridine portion. Conditions can be maintained at, for example, about 100° C., 10-20 bar pressure to enhance regioselectivity. The methylation reaction can be monitored (e.g., via inline monitoring) with NMR and/or ultraviolet-visible (UV-Vis) spectroscopy to facilitate real-time tracking of product formation.

To form a pyrrolidine portion, a second starting material can comprise a pyrrolidine precursor, such as ornithine or a linear amine precursor. The pyrrolidine precursor and a reducing agent (e.g., hydrogen gas) can be passed through a flow reactor to yield the pyrrolidine portion. In various examples, the flow reactor can be packed with a chiral metal catalyst (e.g., Rh or Ir complexes with chiral ligands, such as BINAP or TADDOL (α,α,α′,α′-tetraaryl-2,2-disubstituted 1,3-dioxolane-4,5-dimethanol)). The flow rates, temperature (e.g., 30-50° C.), and/or pressure can be tuned to maximize yield and stereoselectivity.

The pyridine portion and the pyrrolidine portion can be coupled to form the desired 6-methylnicotine. The pyridine portion can be activated by introducing the pyridine portion into a flow reactor with a halogenating agent (e.g., NBS and/or iodine), forming an activated pyridine portion (e.g., 2-methylpyridyl bromide). The activated pyridine portion can be reacted with the pyrrolidine portion in a second flow reactor. A strong base (e.g., NaH or K₂CO₃) can be used to facilitate coupling in a polar aprotic solvent (e.g., acetonitrile and/or DMSO). Inline mixers can facilitate thorough contact of reactants, improving yield and efficiency.

The product comprising 6-methylnicotine can be purified and isolated. Inline separation can comprise passing the reaction mixture through a separation unit (e.g., membrane-based filtration) to remove by-products. The product comprising 6-methylnicotine can be extracted (e.g., via continuous liquid-liquid extraction) to isolate the crude product. The product comprising 6-methylnicotine can be purified via inline chromatography process and/or a crystallization process.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed. Real-time spectroscopy can be used to facilitate and monitor product quality and process optimization.

Formation of 6-methylnicotine via flow chemistry synthesis can have various benefits including precision control (temperature, pressure, and/or residence time can be finely controlled for high regio- and stereoselectivity (e.g., achieving greater than 80% or 90% of the desired isomer and/or enantiomer (i.e., greater than 80% or 90% enantiomeric excess))), scalability (readily scaled by extending reactor lengths or increasing flow rates without altering reaction conditions), efficiency (can produce faster reactions due to enhanced heat and mass transfer in the continuous system), and/or green chemistry (reduced solvent and reagent waste compared to batch processes). Flow chemistry can be advantageous for producing high-value compounds like 6-methylnicotine, such as in pharmaceutical or industrial settings where consistency, scalability, and efficiency are important. This method is highly flexible and adaptable to other synthetic targets.

### Example 7

In various examples, 6-methylnicotine can be formed via solid-phase synthesis, which utilizes immobilized intermediates on a resin to facilitate sequential synthesis steps with high stereochemical control (e.g., greater than 80% or 90% enantiomeric excess) and ease of purification. In this method, intermediates can be covalently attached to a solid support (resin) and undergo sequential chemical transformations. After the synthesis is complete, the final product can be cleaved from the resin.

A starting material can comprise a pyridine precursor, such as a pyridine derivative with a functional group (e.g., carboxylic acid or aldehyde). The pyridine derivative can be attached to a resin (e.g., a commercially available resin, such as Wang resin or Merrifield resin). The resin can be functionalized with a suitable linker that reacts with the pyridine derivative (e.g., ester or imine bond formation). To verify the successful attachment of a pyridine portion to the resin, spectroscopic techniques (e.g., FTIR or UV-Vis spectroscopy) can be used.

Methylation of the immobilized pyridine portion can comprise selective C—H activation. A transition-metal catalyst (e.g., Pd(OAc)₂ with a ligand such as bipyridine) can be used to activate the 2-position of the immobilized pyridine portion. Methyl iodide or dimethyl carbonate can be added to introduce a methyl group at the 2-position of the pyridine portion. Progress can be monitored by cleaving small portions from the resin and analyzing via HPLC or mass spectrometry.

A second starting material can comprise a pyrrolidine precursor, such as an aldehyde or amine derivative. The pyrrolidine precursor can be attached to the resin. For example, for a precursor comprising an amine, an amide bond can be formed with carboxylic acid-functionalized resin (e.g., Wang or trityl resin activated with diisopropylcarbodiimide/1-hydroxy-benzotriazole (DIC/HOBt)). For a precursor comprising an aldehyde, reductive amination can be used on aminomethyl resin (NaBH₃CN or NaBH(OAc)₃) or an imine/oxime can be formed on aldehyde-resin (e.g., BAL linker). The pyrrolidine precursor can be cyclized via reductive amination and/or condensation methods.

The pyridine portion and the pyrrolidine portion can be coupled to form the desired 6-methylnicotine. The pyridine portion can be activated by functionalizing the methylated pyridine portion (e.g., via bromination or chlorination at the 5-position). To achieve nucleophilic substitution, the immobilized pyridine portion can be coupled with the pyrrolidine portion under mild basic conditions (e.g., potassium carbonate or triethylamine).

The product comprising 6-methylnicotine can be cleaved from the resin, e.g., by releasing the final product from the resin using mild acidic (e.g., pH of 4-6.9, 4-6, 5-6.9, or 5.5-6.5) or mild basic (e.g., pH of 7.1-10, 8-10, or 7.5-8.5) conditions, depending on the linker chemistry. For example, trifluoroacetic acid (TFA) can be used for ester linkages or a reducing agent for imine linkages. The product comprising 6-methylnicotine can be extracted with an organic solvent (e.g., ethyl acetate or dichloromethane). The product comprising 6-methylnicotine can be purified via preparative HPLC or recrystallization.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed.

Formation of 6-methylnicotine via solid-phase synthesis can have various benefits including ease of purification (unreacted reagents and by-products can be washed away after each step), automation-friendly (suitable for automated synthesis setups, increasing throughput), high yield (maximize amount of product obtained relative to theoretical maximum, e.g., exceeding 80% or 90% yield) and high purity (purity levels above 90 or 95%) (minimize loss of intermediates and high stereoselectivity due to controlled conditions), and/or versatility (can be adapted for other nicotine derivatives or alkaloid synthesis). This method can be useful for small-scale, high-purity synthesis or for synthesizing libraries of analogs for pharmaceutical research.

### Example 8

In various examples, 6-methylnicotine can be formed via organometallic catalysis with stereoselective control, which uses advanced metal-catalyzed reactions to construct the molecule with high regio- and stereoselectivity. This method utilizes metal catalysts, such as palladium, rhodium, or nickel complexes, to achieve selective methylation and stereocontrol during the synthesis of 6-methylnicotine.

A starting material can comprise a pyridine portion (e.g., pyridine or a pyridine precursor). A palladium catalyst (Pd(OAc)₂) with a suitable ligand (e.g., bipyridine) can be used to activate the C—H bond at the 2-position of the pyridine portion. A methylating agent such as methyl iodide or dimethyl carbonate can be introduced to methylate the pyridine portion at the 2-position. Silver salts (e.g., Ag₂CO₃) can be used as co-catalysts to enhance regioselectivity. Reaction conditions can comprise 100-120° C. in a polar aprotic solvent such as acetonitrile.

A second starting material can comprise a pyrrolidine precursor, such as prochiral imines and/or linear amines. For cyclization via rhodium catalysis, a rhodium complex (e.g., BINAP or (R)-SEGPHOS) can be used. For alternative nickel-catalyzed hydrogenation, a nickel catalyst can be employed during hydrogenation of an imine intermediate to the pyrrolidine portion.

The pyridine portion and the pyrrolidine portion can be coupled to form the desired 6-methylnicotine. The pyridine portion can be activated via halogenation using NBS to form the activated pyridine portion (e.g., 2-methylpyridyl bromide). Suzuki coupling or similar cross-coupling reaction can be used to attach the pyrrolidine portion to the activated pyridine portion. A catalyst comprising a palladium(0) complex (e.g., Pd(PPh₃)₄) can be used for a Suzuki-Miyaura coupling. Nickel-based catalysts can be used for cost-effective alternatives. The reaction conditions for this coupling reaction can comprise mild heating (50-70° C.) in solvents like DMF or tetrahydrofuran (THF).

The product comprising 6-methylnicotine can be purified and isolated. The product comprising 6-methylnicotine can be extracted (e.g., via ethyl acetate or dichloromethane). The product comprising 6-methylnicotine can be purified via silica gel or reverse-phase chromatography. The 6-methylnicotine can be recrystallized from ethanol or methanol to facilitate/maintain high purity.

To verify formation of 6-methylnicotine, NMR spectroscopy, gas chromatography, and/or mass spectrometry can be performed. To determine enantiomeric purity (e.g., enantiomeric excess), chiral HPLC and/or optical rotation measurements can be performed.

Formation of 6-methylnicotine via flow chemistry synthesis can have various benefits including precision (high regioselectivity (e.g., achieving greater than 80% or 90% of the desired isomer) and high stereoselectivity (e.g., greater than 80% or 90% enantiomeric excess) can be achievable using tailored ligands and catalysts), scalability (organometallic reactions can be suitable for industrial-scale synthesis), and/or versatility (the method can allow fine-tuning of reaction conditions for related derivatives). This organometallic route offers a robust and efficient pathway to synthesize 6-methylnicotine with precise control over stereochemistry and regioselectivity, making it advantageous for high-purity and high-yield applications.

### Example 9

In various examples, 6-methylnicotine can be formed via microbial fermentation using engineered metabolic pathways, which uses genetically modified microorganisms to biosynthesize 6-methylnicotine by integrating tailored enzymes and metabolic networks. This method utilizes engineering microbes, such asEscherichia coli(E. coli), Saccharomyces cerevisiae, or Pseudomonas putida, to produce 6-methylnicotine by introducing and optimizing biosynthetic pathways.

To engineer a metabolic pathway, a microbial host can be selected. A robust microorganism can be chosen, likeE. coli, for its ease of genetic manipulation and rapid growth/reproduction. S. cerevisiae can also be used for its ability to handle complex biosynthetic pathways.

Genes encoding enzymes can be introduced for biosynthesis of the pyridine portion (e.g., 2-methylpyridine), pyrrolidine portion synthesis, and/or a coupling reaction between the pyridine portion and the pyrrolidine portion. Pyridine methyltransferase can be used for methylation at the 2-position of the pyridine portion. An isoflavone reductase-like (PIP family) oxidoreductase (e.g., A622), can activate the pyridine precursor (e.g., nicotinic acid derivative). Ornithine decarboxylase can be used to convert ornithine to putrescine (a precursor for pyrrolidine). Pyrrolidine synthase (e.g., an enzyme that creates, or facilitates the creation of pyrrolidine) can be used to cyclize putrescine to form the pyrrolidine portion. Alkaloid-coupling enzyme can be used to facilitate the nucleophilic substitution to form 6-methylnicotine. For example, flavin-containing oxidoreductases can be used for the late condensation/oxidation step linking the pyridine and pyrrolidine (e.g., N-methylpyrrolinium cation) rings.

Strong, inducible promoters (e.g., T7 or GAL promoters) can be used to ensure high expression of biosynthetic genes. Cofactors (e.g., S-adenosylmethionine (SAM) for methylation reactions) can be regenerated by adding enzymes to the reaction. Metabolic flux can be optimized to facilitate channeling of precursors such as ornithine and pyridine toward 6-methylnicotine production. Media optimization can include using a rich medium supplemented with precursors (e.g., pyridine, ornithine) and cofactors. For fermentation conditions, maintain temperature at 30-37° C. and pH of about 7.0 to facilitate microbial growth and enzyme activity. Sufficient aeration for aerobic processes can be provided. Biosynthetic genes can be induced at the appropriate growth phase using inducers (e.g., isopropylthio-β-galactoside (IPTG) or galactose). IPTG is a non-metabolizable lactose analog that can bind the lac repressor (LacI), derepressing the promoter and inducing high-level expression of biosynthetic genes. Galactose can activate Gal4p transcription factor (relieving Gal80p inhibition), strongly inducing GAL-regulated genes. Cells can be grown initially on raffinose (non-repressing), then switched to galactose (2%) for induction which can avoid glucose repression and enable tight, high-expression control for complex eukaryotic pathways.

Cells can be harvested at peak production (e.g., late log phase). Cells can be lysed using mechanical or chemical methods to release 6-methylnicotine. The product can be extracted using an organic solvent like dichloromethane or ethyl acetate. The product comprising 6-methylnicotine can be purified via chromatography techniques such as reverse-phase HPLC. The purified compound can be crystallized to preserve high-quality final product.

To verify formation of 6-methylnicotine, NMR spectroscopy, gas chromatography, and/or mass spectrometry can be performed. To determine enantiomeric purity (e.g., enantiomeric excess), chiral HPLC and/or optical rotation measurements can be performed.

Formation of 6-methylnicotine via microbial fermentation can have various benefits including scalability (readily scalable for industrial production), sustainability (reduces chemical waste and relies on renewable feedstocks), high specificity (genetically-engineered enzymes facilitate high regio- and stereoselective synthesis (e.g., achieving greater than 80% or 90% of the desired isomer and/or greater than 80% or 90% enantiomeric excess)), and/or cost-effectiveness (reduces the need for expensive chemical reagents and catalysts). This microbial fermentation process can be suitable for large-scale production where sustainability, cost-efficiency, and high stereochemical purity are important.

### Example 10

In various examples, 6-methylnicotine can be formed via enzymatic cascade synthesis, which uses engineered enzymes to produce 6-methylnicotine in a highly selective and environmentally-friendly (e.g., green chemistry) process. This method utilizes the specificity of enzymes to perform sequential reactions in a controlled and efficient manner.

A starting material can comprise a pyridine portion (e.g., pyridine or a pyridine precursor). An enzyme (e.g., engineered pyridine methyltransferase) can be used to methylate the pyridine portion. A naturally-occurring methyltransferase can be modified to selectively methylate the 2-position of the pyridine portion. Such modification can occur via mutagenesis (error-prone PCR) or site-saturation mutagenesis on active-site residues, which can be followed by high-throughput screening for activity on pyridine and regioselectivity (e.g., via HPLC/MS assay for 2-methylpyridine formation). Examples can include evolving halide methyltransferases for altered alkyl specificity and/or O-methyltransferases for switched regioselectivity (para vs. meta). SAM can be used as a methyl donor and cofactor for methylation. In various examples, modification of methyltransferase can occur by using crystal structures or homology models to target substrate-binding pocket residues, enlarging/narrowing the cavity, and/or introducing H-bonding/steric groups to favor pyridine orientation for C2 (2-position) attack.

The pyridine portion, SAM, and the engineered methyltransferase can be combined in a buffered solution. The solution pH (e.g., 7.0-8.0) and temperature (30-37° C.) can be optimized for enzyme activity. An auxiliary enzyme, such as SAM synthetase, can be used to regenerate SAM in situ, thus reducing waste and/or expense.

A second starting material can comprise a pyrrolidine precursor, such as L-ornithine. An enzyme (e.g., aminotransferase) can be used to convert (cyclize) the pyrrolidine precursor to a pyrrolidine ring portion. The pyrrolidine precursor (e.g., ornithine and a keto acid such as α-ketoglutarate) and the enzyme can be provided in a buffered solution, the conditions for which can be optimized for the chosen enzyme (e.g., pH 7.0-8.0 and 25-37° C.).

The pyridine portion and the pyrrolidine portion can be coupled to form the desired 6-methylnicotine. An enzyme capable of catalyzing the nucleophilic substitution of the pyridine portion with the pyrrolidine portion can be used (an engineered alkaloid synthase enzyme and/or a bifunctional enzyme). The pyridine portion and the pyrrolidine portion can be combined in the presence of the coupling enzyme. Such an enzyme can comprise A622 (e.g., an isoflavone reductase-like (PIP family) oxidoreductase, which can activate/reduce the pyridine precursor (e.g., forms a dihydronicotinic acid intermediate)), berberine bridge enzyme-like proteins (BBLs) (e.g., flavin-containing oxidoreductases (e.g., BBLa, BBLb, etc.), which can perform late-stage oxidation/aromatization after ring coupling), a UDP-glucosyltransferase (UGT) for cryptic glucosylation activation, and/or a β-glucosidase (β-GD) for deglucosylation. In various examples, forming nicotine or a derivative thereof (e.g., 6-methylnicotine) can comprise UGT, A622, BBLa, and/or β-GD. Cofactors or coenzymes can be provided to facilitate the reaction.

The product comprising 6-methylnicotine can be purified and isolated. The product comprising 6-methylnicotine can be extracted using an organic solvent like ethyl acetate. The product comprising 6-methylnicotine can be purified via column chromatography and/or preparative HPLC. The 6-methylnicotine can be recrystallized from ethanol or methanol to facilitate/preserve high purity.

To verify formation of 6-methylnicotine, NMR spectroscopy, gas chromatography, and/or mass spectrometry can be performed. To determine enantiomeric purity (e.g., enantiomeric excess), chiral HPLC can be performed (e.g., to confirm desired S-enantiomer formed).

Formation of 6-methylnicotine via enzymatic cascade synthesis can have various benefits including high selectivity (enzymes facilitate regio- and stereoselectivity, reducing the need for further resolution), sustainability (environmentally-friendly, as enzymes operate under mild conditions with minimal hazardous waste), efficiency (cascading enzymes can reduce the number of reaction steps and intermediate purifications), and/or scalability (readily adapted for industrial production with optimized enzyme formulations). This process can be suitable for pharmaceutical and fine chemical applications, especially when high stereochemical purity is important.

### Example 11

In various examples, 6-methylnicotine can be formed via a cascade reaction, which combines multiple reaction steps into a single pot (i.e., reaction vessel) or sequence to streamline the synthesis of 6-methylnicotine. This method can increase efficiency and reduce intermediate handling. A cascade or tandem reaction can involve creating both the pyridine portion and the pyrrolidine portion, followed by coupling them in a streamlined sequence. This approach leverages carefully chosen catalysts and conditions to achieve regio- and stereoselectivity.

A starting material can comprise a pyridine portion (e.g., pyridine or a pyridine precursor). A methylating agent (e.g., methyl iodide or dimethyl carbonate) can be used in the presence of a catalyst like Cu(I) salts or Lewis acids to methylate the pyridine portion. The reaction conditions can be optimized to selectively methylate the 2-position of the pyridine portion, potentially using directing groups or additives like boron trifluoride (BF₃). The pyridine portion (e.g., 2-methylpyridine) can be optionally brominated at the 5-position using NBS (to activate the pyridine) for subsequent coupling steps.

A second starting material can comprise a pyrrolidine precursor, such as L-proline and/or a linear amine precursor. In a one-pot cyclization, the pyrrolidine precursor can be combined with a suitable reagent (e.g., aldehyde or ketone) and reduced under catalytic conditions (e.g., using hydrogen gas with a chiral metal catalyst or sodium borohydride with organocatalysis) to form the pyrrolidine portion.

The pyridine portion and the pyrrolidine portion can be coupled to form the desired 6-methylnicotine. In the same pot or sequentially, the pyridine portion can be activated (e.g., via halogenation or triflate formation), and the pyrrolidine portion can be added under basic conditions (e.g., using K₂CO₃ or NaH) to perform the nucleophilic substitution. A polar aprotic solvent (e.g., DMF and/or DMSO) can be used to facilitate both the functionalization/activation of the pyridine portion and the substitution reaction.

The product comprising 6-methylnicotine can be purified and isolated. The product comprising 6-methylnicotine can be extracted using an organic solvent such as ethyl acetate. The product comprising 6-methylnicotine can be purified via crystallization from a suitable solvent, column chromatography, and/or preparative HPLC to remove impurities.

If any racemization occurs, the enantiomers can be separated using chiral chromatography. Chiral ligands and/or additives can be used to avoid racemization during synthesis.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed (e.g., to confirm desired S-enantiomer formed).

Formation of 6-methylnicotine via cascade reaction can have various benefits including efficiency (can combine multiple reaction steps into one pot or vessel, reducing intermediate isolation and purification), sustainability (can minimize waste and resource usage by integrating reactions), and/or scalability (simplifies the process for industrial-scale synthesis). This process offers a streamlined and cost-effective approach to synthesizing 6-methylnicotine, particularly for large-scale production where reducing time and resources is desired.

### Example 12

In various examples, 6-methylnicotine can be formed via photo-redox catalysis combined with enantioselective synthesis, which leverages light-driven reactions and chiral catalysts to achieve the regioselective and stereoselective synthesis of 6-methylnicotine.

A starting material can comprise a pyridine portion (e.g., pyridine or a pyridine precursor). The pyridine portion can be photo-redox functionalized. A visible-light photo-redox catalyst can be used, such as [Ru(bpy)₃]Cl₂, and/or an organic photocatalyst (e.g., eosin Y or 9-mesityl-10-methylacridinium). A methyl radical precursor (e.g., trimethylsilylmethyl chloride or acetophenone derivatives) can be added. The reaction can be irradiated with visible light (e.g., LEDs at 450 nm) to generate methyl radicals. For example, reacting a protonated pyridine (or pyridine N-oxide), a methyl radical source (e.g., acetic acid, persulfate (e.g., (NH₄)₂S₂O₈), methanol/TBHP, dimethyl sulfoxide (DMSO), and/or trimethyl orthoformate), and a photocatalyst (e.g., Ir(ppy)₃, [Ru(bpy)₃]Cl₂, or organic dye like Eosin Y) under blue LED irradiation at about room temperature. The photocatalyst can generate ·CH₃ radicals via oxidative or reductive quenching. The nucleophilic methyl radical can be added to the protonated/activated pyridine (electron-deficient), preferentially at the 2- and/or 4-positions due to electronic bias. The methyl radical can be reacted with the pyridine portion under controlled conditions (e.g., polar aprotic (MeCN, DMF, or DMSO), include water (e.g., 5-20% wt) or co-solvents for selectivity, about room temperature, inter environment, for any suitable duration (e.g., 12-24 hours)) to achieve methylation of the pyridine portion at the 2-position. The pyridine portion (e.g., 2-methylpyridine) can be purified using column chromatography.

A second starting material can comprise a pyrrolidine precursor, such as prochiral imine and/or amino alcohol derivatives. A chiral transition metal catalyst (e.g., Rh or Ir complexes with BINAP or TADDOL-derived ligands) can be used in the presence of a reducing agent (e.g., hydrogen gas or formic acid). The precursor can be cyclized into the pyrrolidine portion.

The pyridine portion and the pyrrolidine portion can be coupled to form the desired 6-methylnicotine. The pyridine portion can be activated, for example, by halogenation (e.g., bromination or iodination using NBS or iodine monochloride (ICl)). The halogenated pyridine portion can be reacted with the pyrrolidine portion in the presence of a strong base (e.g., NaH or K₂CO₃). A polar aprotic solvent such as DMSO or acetonitrile can be used to enhance the reaction efficiency.

The product comprising 6-methylnicotine can be purified and isolated. The product comprising 6-methylnicotine can be extracted using an organic solvent (e.g., ethyl acetate or dichloromethane). The product comprising 6-methylnicotine can be purified via silica gel and/or reverse-phase chromatography. The 6-methylnicotine can be crystallized to facilitate/preserve high purity.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed (e.g., to confirm desired S-enantiomer formed).

Formation of 6-methylnicotine via photo-redox catalysis can have various benefits including mild reaction conditions (light-driven reactions occur at room temperature, reducing energy consumption), regioselectivity and enantioselectivity (controlled by the photocatalyst and/or chiral catalyst, ensuring precise synthesis), and/or sustainability (can avoid harsh reagents and relies on visible light as a sustainable energy source). This process can provide a highly efficient, selective, and scalable route to synthesizing 6-methylnicotine, making it suitable for applications where green chemistry principles are prioritized.

### Example 13

In various examples, 6-methylnicotine can be formed via electrochemical synthesis combined with stereoselective catalytic processes, which leverages electrochemistry to functionalize pyridine derivatives and stereoselective catalysts to introduce chirality.

A starting material can comprise a pyridine portion (e.g., pyridine or a pyridine precursor). The pyridine portion can be methylated. An electrochemical cell can be used with the pyridine portion as the substrate. A methyl donor (e.g., dimethyl carbonate or methyl iodide) can be introduced into the electrolyte solution. A controlled voltage can be applied to selectively methylate the 2-position of the pyridine portion, facilitated by an anodic oxidation process. Conditions (e.g., current density, solvent, and electrolyte) can be optimized to achieve regioselective 2-methylpyridine formation. For example, conditions can comprise a solvent (e.g., comprising acetonitrile or DMF (e.g., including acetic acid)), electrolyte (e.g., NaOAc or Tetrabutylammonium Tetrafluoroborate (TBABF4)), a (undivided) cell with Pt or carbon anode, current density: 10-50 mA/cm², voltage: 2-4 V (e.g., constant potential vs Ag/AgCl); temperature: 20-40° C.; inert atmosphere; and/or charge: 2-4 F/mol). As another example, for a PD-catalyzed electro-methylation with MeBF₃K, the conditions can include a solvent (e.g., Trifluoroethanol, AcOH, and/or H₂O mixture); a catalyst: Pd(OAc)₂ (e.g., including bipyridine ligand); an electrolyte; Pt—Pt cell; current density: 5-20 mA/cm²; temperature: about room temperature; charge: 1.5-3 F/mol. As another example, for a mediated anodic with peroxide/acid, the conditions can include a solvent (e.g., MeCN and/or TFA); a methyl source (e.g., DMSO and/or AcOH (e.g., including persulfate); current density: 20 mA/cm²; voltage: 1.5-2.5 V vs SCE; temperature: 0-25° C.

A second starting material can comprise a pyrrolidine precursor, such as linear amines and/or cyclic imines. A pyrrolidine precursor can be oxidized to form a reactive iminium ion. The iminium ion can be reduced electrochemically (e.g., in the presence of a chiral catalyst (e.g., a chiral phosphoric acid or organocatalyst)), which introduces the methyl group.

The pyridine portion and the pyrrolidine portion can be coupled to form the desired 6-methylnicotine. The pyridine portion can be activated, for example, by an electrochemical halogenation process. To do so, a current can be passed through the solution containing the pyridine portion and a halogen source (e.g., NBS or bromine). For nucleophilic substitution, the halogenated pyridine portion can be combined with the pyrrolidine portion in the presence of a base (e.g., NaH or K₂CO₃) under mild heating in a polar aprotic solvent.

The product comprising 6-methylnicotine can be purified and isolated. The product comprising 6-methylnicotine can be optionally electrochemically purified by employing a paired electrolysis setup to selectively oxidize impurities or side products, leaving the desired product untouched (i.e., unreacted). The product comprising 6-methylnicotine can be extracted using an organic solvent (e.g., ethyl acetate or dichloromethane). The product comprising 6-methylnicotine can be purified via preparative HPLC and/or column chromatography. The 6-methylnicotine can be crystallized to facilitate/preserve high purity and/or stability.

To verify formation of 6-methylnicotine, NMR spectroscopy, gas chromatography, and/or mass spectrometry can be performed. To determine enantiomeric purity (e.g., enantiomeric excess), chiral HPLC and/or optical rotation measurements can be performed (e.g., to confirm desired S-enantiomer formed).

Formation of 6-methylnicotine via electrochemical synthesis can have various benefits including green chemistry (can reduce reliance on harsh chemicals and promote sustainable practices), high regioselectivity (electrochemical methods can offer precise control over the site of functionalization), scalability (electrochemical setups can be adapted for both lab and industrial scales), and/or enantioselectivity (achieved through the use of chiral catalysts during reduction). This process integrates modern electrochemical techniques with stereoselective catalysis, providing a sustainable, efficient, and scalable approach to synthesizing 6-methylnicotine.

### Example 14

In various examples, 6-methylnicotine can be formed via enantioselective organocatalytic synthesis, which can rely on modern catalytic systems and stereoselective strategies. Such a process may not include the use of biological components. This approach focuses on using small organic molecules as catalysts to achieve high stereochemical control.

A starting material can comprise a pyridine portion (e.g., pyridine or a pyridine precursor). The 2-position of the pyridine portion can be selectively lithiated using tert-butyllithium. The reaction temperature can be about −78° C. Methyl iodide can be introduced to attach a methyl group at the 2-position of the pyridine portion. A solvent can comprise diethyl ether, hexanes, and/or THF. The reaction(s) can be run under inert environment at any suitable temperature (e.g., about −78° C.). The product comprising the pyridine portion (e.g., 2-methylpyridine) can be distilled and/or recrystallized.

A second starting material can comprise a pyrrolidine precursor, such as L-proline and/or an achiral precursor. An organocatalyst, such as a chiral imidazolidinone derivative, can be used to facilitate the cyclization of an appropriate linear precursor into a pyrrolidine ring portion. Enantioselective methylation of the pyrrolidine portion can comprise performing a catalytic enantioselective reductive amination using a chiral organocatalyst (e.g., chiral imidazolidinone derivatives such as proline-derived imidazolidinone and/or MacMillan catalyst, L-proline and modified proline catalysts such as L-proline and/or proline amides and derivatives, chiral phosphoric acids (CPA) such as BINOL-phosphoric acids and/or TRIP (2,4,6-triisopropylphenyl)-substituted CPA, chiral Brønsted bases such as Sparteine-derived catalysts, and/or chiral secondary amines such as Jørgensen-Hayashi catalyst) and a methyl donor (e.g., formaldehyde or paraformaldehyde).

The pyridine portion and the pyrrolidine portion can be coupled to form the desired 6-methylnicotine. The pyridine portion can be activated, for example, by a halogenation process. To do so, the pyridine portion can be brominated and/or chlorinated at the 5-position using a halogenating agent (e.g., SOCl₂ or NBS). For nucleophilic substitution, the activated pyridine portion derivative can be reacted with the pyrrolidine portion in the presence of a strong base (e.g., sodium hydride or potassium tert-butoxide). A polar aprotic solvent (e.g., a strong base (e.g., sodium hydride or potassium tert-butoxide). A polar aprotic solvent (e.g., DCM, toluene, MTBE, DMF and/or acetonitrile) can be used to facilitate the reaction.

The product comprising 6-methylnicotine can be purified and isolated. The product comprising 6-methylnicotine can be extracted using an organic solvent (e.g., ethyl acetate or dichloromethane). The product comprising 6-methylnicotine can be purified via silica column chromatography or preparative HPLC. The 6-methylnicotine can be recrystallized from a solvent system that favors the S-enantiomer, such as ethanol or methanol.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed (e.g., to confirm desired S-enantiomer formed).

Formation of 6-methylnicotine via enantioselective organocatalytic synthesis can have various benefits including organocatalysis (can eliminate or decrease reliance on metal-based catalysts, making the process greener and more sustainable), high enantioselectivity (achieved through the use of chiral organocatalysts), and/or scalability (suitable for both lab-scale and industrial production). This process can be advantageous when high stereochemical purity is required for pharmaceutical or fine chemical applications. It offers a robust and scalable approach to synthesizing 6-methylnicotine.

### Example 15

In various examples, 6-methylnicotine can be formed via total synthesis with asymmetric induction, which incorporates synthetic strategies to achieve both regioselectivity and stereoselectivity for the synthesis of 6-methylnicotine.

A starting material can comprise a pyridine portion (e.g., pyridine or a pyridine precursor, such as 2-chloropyridine). The pyridine portion can be treated with a base (e.g., n-butyllithium (n-BuLi) and/or tert-butyllithium) to generate a lithiated intermediate. Solvent for such a reaction can comprise diethyl ether, THF, and/or hexanes. The reaction temperature can be about at −78° C. Methyl iodide (CH₃I) can be introduced to selectively attach a methyl group at the 2-position of the pyridine portion (solvent can comprise, e.g., diethyl ether, THF, and/or hexanes). The 5-position of the pyridine portion (e.g., 2-methylpyridine) can be brominated using NBS for future coupling flexibility.

A second starting material can comprise a pyrrolidine precursor, such as L-proline and/or an equivalent chiral amine. The pyrrolidine precursor can be cyclized via an intramolecular condensation reaction or using a reductive amination strategy (e.g., NaBH₃CN and/or catalytic hydrogenation), and/or via condensation/reduction. Asymmetric hydrogenation can be used with a chiral catalyst (e.g., Rh-BINAP or Ru-SYNPHOS) to selectively introduce the methyl group at the nitrogen of the pyrrolidine portion. The N-methyl group can be introduced stereoselectively via asymmetric hydrogenation of a cyclic imine intermediate (Δ¹-pyrrolinium salt) using a chiral catalyst such as Rh-(R)-BINAP or Ru-(R)-SYNPHOS under H₂ pressure.

The pyridine portion and the pyrrolidine portion can be coupled to form the desired 6-methylnicotine. The pyridine portion can be activated, for example, by a halogenation process. To do so, the pyridine portion can be brominated (e.g., using NBS) to form a reactive intermediate (e.g., 2-methylpyridyl bromide). For nucleophilic substitution, the activated pyridine portion derivative can be reacted with the pyrrolidine portion in the presence of a strong base (e.g., sodium hydride or potassium tert-butoxide). A polar aprotic solvent (e.g., DCM, toluene, MTBE, DMF and/or acetonitrile) can be used to facilitate the reaction.

The product comprising 6-methylnicotine can be purified and isolated. The product comprising 6-methylnicotine can be extracted using an organic solvent (e.g., ethyl acetate or dichloromethane). The product comprising 6-methylnicotine can be purified via silica column chromatography or preparative HPLC. The 6-methylnicotine can be recrystallized from a solvent system that favors the S-enantiomer, such as ethanol or methanol.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed (e.g., to confirm desired S-enantiomer formed).

### Example 16

In various examples, 6-methylnicotine can be formed via biosynthesis using a cell factory model. Creating a cell factory model involves engineering microorganisms (e.g., E. coli, Saccharomyces cerevisiae, and/or Pseudomonas putida) to produce 6-methylnicotine by introducing biosynthetic pathways, optimizing metabolic flux, and ensuring stereochemical control. The process can include tailored steps for 2- or 6-methyl functionalization.

The host microorganism can be engineered for the biosynthesis. First, a host can be selected. For example, E. coli can be selected because of its fast growth/reproduction, ease of genetic modification, high yield of heterologous proteins; S. cerevisiae can be selected because of its efficient alkaloid pathway expression; and/orpseudomonas putida can be selected because of its robust metabolism for aromatic compound production.

The genes for the following enzymes can be introduced and/or expressed: quinolinate synthase (can convert L-aspartate and dihydroxyacetone phosphate into quinolinic acid, a precursor for nicotinic acid); quinolinate phosphoribosyltransferase (QPRT) (can convert quinolinic acid into nicotinic acid mononucleotide (NAMN)); 6-Methyltransferase (engineered) (can catalyze selective methylation at the 6-position of nicotinic acid); ornithine decarboxylase (ODC) (can convert L-ornithine to putrescine, a precursor for the pyrrolidine ring); pyrrolidine synthase (engineered) (can cyclize putrescine into pyrrolidine); and/or alkaloid coupling enzyme (engineered) (can join the pyridine portion backbone with the pyrrolidine portion to form 6-methylnicotine).

The synthesis (e.g., fermentation) process can be divided into modules for pyridine portion biosynthesis, pyrrolidine portion production, and a coupling reaction. The pyridine portion biosynthesis module can comprise introducing genes for quinolinate synthesis and methyltransferase into the host genome. L-aspartate and dihydroxyacetone phosphate can be converted into quinolinic acid, which can be converted into nicotinic acid (e.g., via QPRT). The methyltransferase can catalyze selective methylation of nicotinic acid at the 6-position. The nicotinic acid can be decarboxylated to form the pyridine portion (e.g., 2-methylpyridine).

The pyrrolidine portion biosynthesis module can comprise integrating genes for ornithine decarboxylase and pyrrolidine synthase. With a pyrrolidine precursor comprising L-ornithine, ODC can convert L-ornithine to putrescine. Pyrrolidine synthase (engineered) can cyclize putrescine into the pyrrolidine portion. The coupling reaction module can comprise expressing alkaloid coupling enzymes for final product assembly (e.g., coupling the pyridine portion and the pyrrolidine portion to form 6-methylnicotine). Pathways can be incorporated for recycling essential cofactors such as SAM (for methylation reactions) and/or (nicotinamide adenine dinucleotide phosphate) NADPH (for decarboxylation and reduction steps). Transporter proteins can be expressed for efficient uptake of precursors (e.g., ornithine) and secretion of 6-methylnicotine. For the uptake of precursors, in E. coli, PotE (putrescine-ornithine antiporter) can facilitate ornithine import via antiport with putrescine or H+; ArgP (arginine/ornithine permease) can enable proton-symport uptake, enhancing intracellular levels for pathway flux. For the uptake of precursors, in S. cerevisiae, Gap1 (general amino acid permease) or Ort1 (specific ornithine transporter) can use proton-gradient-driven symport to actively import ornithine, optimizing availability in engineered strains. For section of 6-methylnicotine in E. coli, AtDTX1 (MATE family from Arabidopsis) can export alkaloids via H+ antiport; AcrAB-TolC (RND efflux pump) can use proton motive force for ATP-independent secretion, reducing toxicity and improving yield. For section of 6-methylnicotine in S. cerevisiae, PDR5 or Snq2 (ABC transporters) can employ ATP hydrolysis for active efflux of alkaloids; JAT1-like (MATE from tobacco) can facilitate vacuolar or plasma membrane export via ion exchange.

A fermentation process can be set up including a culture medium (e.g., using a defined medium optimized for precursor availability (e.g., glucose, ammonium, and ornithine)); process parameters such as temperature (optimize for host (e.g., 30° C. for E. coli or 25-30° C. for S. cerevisiae)), pH (maintain pH of about 7.0 for enzyme activity), and/or aeration (facilitate sufficient oxygenation for aerobic metabolism); and/or induction (inducing biosynthetic gene expression with IPTG (for E. coli) or galactose (for S. cerevisiae) at mid-log phase).

Cells can be harvested at peak production phase (late log or early stationary). Cells can be lysed using mechanical or chemical methods to release intracellular 6-methylnicotine. Lysis can include sonication by using ultrasonic waves (e.g., 20-50 kHz, 5-10 min cycles on ice) to disrupt cell membranes via cavitation, bead beating (e.g., agitating cells with glass beads (0.1-0.5 mm) in a vortex mixer or bead mill (e.g., 5 -15 min at 4,000-6,000 rpm)), french press or high-pressure homogenization (passing cell suspension through a narrow valve under high pressure (e.g., 10,000-20,000 psi, 2-3 passes)), and/or freeze-thaw cycles (e.g., repeated freezing (e.g., liquid N₂ or −80° C.) and thawing (e.g., in 37° C. water bath, 3-5 cycles)). Chemical lysis can comprise cold methanol extraction (e.g., quenching and lysing with 50-60% cold methanol (e.g., −48° C., 5-10 min incubation)), boiling ethanol (e.g., suspending cells in 75-100% ethanol and boiling (e.g., 95-100° C., 3-5 min), lysozyme treatment (e.g., adding lysozyme (e.g., 1-5 mg/mL in Tris-EDTA buffer, 30 min at 37° C.) followed by detergent (e.g., 1% Triton X-100) for complete lysis, and/or hot water extraction (e.g., incubating in hot water (e.g., 80-100° C., 5-10 min).

The product comprising 6-methylnicotine can be extracted using an organic solvent (e.g., ethyl acetate or dichloromethane), and/or purified using preparative HPLC and/or silica gel chromatography.

Metabolic engineering and optimization can take place. For example, for enzyme engineering, directed evolution and/or rational design can be used to enhance activity and specificity of key enzymes (e.g., 6-methyltransferase, pyrrolidine synthase). For metabolic flux analysis, carbon flux can be redirected toward the desired product by overexpressing pathway enzymes and/or knocking out competing pathways. For dynamic regulation, regulatory circuits can be introduced to dynamically adjust enzyme expression based on cell growth phase.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed (e.g., to confirm desired S-enantiomer formed). Yield of 6-methylnicotine can be quantified via liquid chromatograph-mass spectrometry (LC-MS) and/or gas chromatograph-mass spectrometry (GC-MS).

Formation of 6-methylnicotine via a cell factory approach can have various benefits including sustainability (produces 6-methylnicotine from renewable feedstocks under mild conditions), scalability (readily scaled in bioreactors for industrial production), high selectivity (enzymatic reactions facilitate regio- and stereoselectivity), and/or reduced costs (minimizes the use of expensive chemical reagents and waste generation). This process offers a sustainable, scalable, and highly selective pathway to produce 6-methylnicotine, suitable for pharmaceutical and industrial applications.

### Example 17

In various examples, 6-methylnicotine can be formed via biosynthesis using minimally engineered microbial host. A minimally engineered microbial host refers to a microorganism (e.g., E. coli or S. cerevisiae) modified with a limited number of genetic interventions—typically introducing or overexpressing only 4-6 key heterologous genes (e.g., QPRT, engineered methyltransferase, ODC, engineered pyrrolidine synthase, and coupling enzymes)—while relying on the host's native metabolism for precursor supply, cofactor regeneration, and basic flux. This contrasts with heavily engineered strains requiring extensive knockouts, multi-gene cassettes, or synthetic scaffolds, improving robustness, reducing off-target effects, and simplifying regulatory approval. 6-methylnicotine can be readily produced via biosynthesis using a minimally engineered microbial host such as E. coli or Saccharomyces cerevisiae, with a simplified and modular pathway. The biosynthetic pathway can comprise steps of methylation of nicotinic acid to produce 6-methylnicotinic acid; decarboxylation of 6-methylnicotinic acid to form the pyridine portion (e.g., 2-methylpyridine); production of the pyrrolidine portion from L-ornithine; and/or coupling of the pyridine portion and the pyrrolidine portion.

The host microorganism can be engineered for the biosynthesis. First, a host can be selected. For example, E. coli can be selected because of its fast growth/reproduction, well-characterized metabolism, and/or efficient enzyme expression; S. cerevisiae can be selected because a eukaryotic environment may be better suited for complex alkaloid biosynthesis.

For the biosynthetic process, minimal pathway genes can be introduced. For methylnicotinic acid synthesis, QPRT (can convert quinolinic acid into nicotinic acid mononucleotide (NaMN)), and/or 6-Methyltransferase (engineered) (can catalyze selective methylation at the 6-position of nicotinic acid, producing 6-methylnicotinic acid. can be used. 6-methylnicotinic acid can undergo decarboxylation to form the pyridine portion (e.g., 2-methylpyridine). For pyrrolidine portion biosynthesis, ODC (converts L-ornithine to putrescine, a precursor for the pyrrolidine portion) and/or pyrrolidine synthase (engineered) (cyclizes putrescine into the pyrrolidine portion) can be used. For coupling, an alkaloid coupling enzyme (engineered) (joins the pyridine portion and the pyrrolidine portion to form 6-methylnicotine) can be used.

Feedstock requirements for this process can comprise precursors including quinolinic acid and/or L-aspartate as a precursor for nicotinic acid and/or L-ornithine for pyrrolidine portion biosynthesis; and/or cofactors including SAM for methylation; and/or ATP and/or NADPH for energy and reduction requirements.

A fermentation process can be set up including culture conditions, such as a culture medium (e.g., a rich or defined medium comprising glucose and amino acids (e.g., L-ornithine)), process parameters such as temperature (about 30° C. for E. coli; 25-30° C. for S. cerevisiae), pH (maintain pH ~7.0 for enzyme activity); and/or induction of gene expression (using an inducible promoter system (e.g., IPTG for E. coli) to express biosynthetic genes at mid-log growth phase). The fermentation process can be similar to, or the same as, that described in Example 16, above.

Cells can be lysed using sonication or chemical lysis to release intracellular products. The product comprising 6-methylnicotine can be extracted using an organic solvent (e.g., ethyl acetate or dichloromethane), and/or purified using a single chromatography step (e.g., silica gel or HPLC) to isolate 6-methylnicotine.

To verify formation of 6-methylnicotine, NMR spectroscopy, gas chromatography, and/or mass spectrometry can be performed. To determine enantiomeric purity (e.g., enantiomeric excess), chiral HPLC and/or polarimetry can be performed (e.g., to confirm desired S-enantiomer formed). Yield of 6-methylnicotine can be quantified via liquid chromatograph-mass spectrometry (LC-MS) and/or gas chromatograph-mass spectrometry (GC-MS).

Formation of 6-methylnicotine via biosynthesis using minimally engineered microbial host can have various benefits including minimal engineering (can require only 4-5 key enzymes, reducing complexity and improving system robustness), easily available precursors (quinolinic acid, nicotinic acid, and L-ornithine are commercially available or easy to produce), scalability (simple fermentation setup suitable for scale-up), and/or cost-effectiveness (fewer enzymes and steps mean reduced operational costs). This process offers a sustainable, scalable, and highly selective pathway to produce 6-methylnicotine, suitable for pharmaceutical and industrial applications.

An example production workflow of this example can comprise inoculation (engineered E. coli in a defined medium); feeding (supplement with glucose, quinolinic acid, and/or L-ornithine); induction (add IPTG to induce biosynthetic enzymes); fermentation (allow the culture to grow for 24-48 hours); and/or harvest (collect cells and extract 6-methylnicotine from the lysate). This streamlined approach can minimize the complexity of the pathway, relying on well-characterized enzymes and readily available precursors.

### Example 18

In various examples, 3-cyano-6-methylpyridine can be a precursor used to couple to a pyrrolidine portion to form 6-methylnicotine. Biosynthesis of 3-cyano-6-methylpyridine can comprise beginning with a pyridine portion (e.g., pyridine and/or nicotinic acid), which can be methylated. Methylation of the pyridine portion at the 2-position (to form 2-methylpyridine), or nicotinic acid at the 6-position (to form 6-methylnicotinic acid), can be completed by any suitable process, such as those discussed herein. 6-methylnicotinic acid can be decarboxylated to form the pyridine portion (e.g., 2-methylpyridine). A cyano group can be added at the 5-position of 2-methylpyridine to form 3-cyano-6-methylpyridine. These reactions can be facilitated/mediated by specific methyltransferase and cyanosynthase enzymes. The nitrile group on the 3-cyano-6-methylpyridine can be reduced to form a reactive intermediate for coupling with the pyrrolidine moiety. In various examples, formation of 3-cyano-6-methylpyridine (e.g., 5-cyano-2-methylpyridine) can occur via nitrile activation/reduction to an imine or aldehyde intermediate (e.g., forming a reactive pyridyl-methylimine or aldehyde at the 5-position for Mannich-like or reductive amination coupling, analogous to myosmine intermediates in nicotine synthesis). For methylation, chemical (e.g., radical Minisci with ·CH₃ sources) or engineered enzymatic methods can be used (hypothetical SAM-dependent methyltransferase tuned for C6 on nicotinic acid). Thermal/Cu-catalyzed from 6-methylnicotinic acid to 2-methylpyridine can occur for decarboxylation. For addition of cyano group at 5-position: a chemical method can comprise activating as 2-methylpyridine N-oxide, then using TMSCN or Zn(CN)₂ with reagents (e.g., Tf₂O or cyanating agents) for Reissert-Henze-type cyanation at C2 of the oxide (corresponding to 6-position); and/or a directed C—H cyanation (e.g., Cu/Pd-catalyzed with N-cyano succinimide) or Sandmeyer on 5-amino-2-methylpyridine can occur. For nitrile reduction and coupling, partial reduction of the nitrile with DIBAL-H or Raney Ni/H₂ to the aldehyde (5-formyl-2-methylpyridine) can occur, and the aldehyde/imine can react with N-methyl-Δ¹-pyrrolinium (from pyrrolidine portion) in Mannich-type condensation, followed by reduction/aromatization to attach at the 5-position, yielding 6-methylnicotine scaffold.

The processes and steps in these examples of 6-methylnicotine formation can be used to form nicotine and/or other nicotine derivatives, for example by the precursors comprising (or being used to form) pyridine derivatives and moieties and pyrrolidine derivatives and moieties that, when coupled, form nicotine or the desired nicotine derivative. Thus, the disclosure of these examples can also apply to the synthesis of nicotine.

For processes disclosed herein for forming nicotine derivatives (e.g., 6-methylnicotine) in the above examples, the extracted and/or purified product can be substantially or entirely free and devoid of nicotine (“substantially” in this context means having less than 5% or 2% or 1% nicotine).

### Nicotine

In various examples, biosynthesis of nicotine can comprise starting with a polyamine precursor (e.g., a pyridine portion and/or a pyrrolidine portion). For example, putrescine can be formed from ornithin and/or arginine. Ornithin decarboxylase (ODC) can be used to convert ornithin into putrescine. Putrescine can be methylated by putrescine N-methyltransferase (PMT) to form N-methylputrescine. N-methylputrescine can be deaminated to form N-methylpyrrolinium cation. For example, an enzyme comprising N-methylputrescine oxidase (MPO) can oxidatively deaminate N-methylputrescine to form the N-methylpyrrolinium cation, a pyrrolidine intermediate.

Another starting compound in the biosynthesis of nicotine can comprise nicotinic acid. The nicotinic acid can be formed for nicotine biosynthesis via any suitable process (e.g., nicotinamide adenine dinucleotide (NAD) degradation). Nicotinic acid can be converted into a nicotinic acid derivative (e.g., nicotinic acid mononucleotide (NaMN)). The nicotinic acid derivative can be modified to form a pyridine portion.

To form nicotine, the N-methylpyrrolinium cation and nicotinic acid derivative can react in a condensation rection catalyzed by a quinolinate synthase-like enzyme.

In various examples, more generally, the biosynthesis of nicotine can begin with a pyridine portion and a pyrrolidine portion. The pyridine portion and the pyrrolidine portion can be coupled to form the desired nicotine compound.

The desired product can be extracted (e.g., by a liquid-liquid extraction using ethyl acetate and/or dichloromethane (DCM)) and/or isolated (e.g., via distillation). The product can be purified by any suitable method, such as silica gel chromatography, column chromatography, high performance chromatography, recrystallization, and/or the like. Resulting nicotine can be crystallized for stability and high purity.

To verify that the product comprises the desired nicotine, product verification can be conducted through any suitable method. For example, nuclear magnetic resonance (NMR) spectroscopy (e.g., 1H, 13C), fourier transform infrared (FTIR) spectroscopy, gas chromatography, and/or mass spectrometry can be utilized for structure elucidation. To determine the enantiomeric purity (e.g., enantiomeric excess) of the nicotine, the product can be analyzed using chiral column high-performance liquid chromatography (HPLC), polarimetry, or any other suitable method.

The following examples recite a series of steps for the formation (e.g., synthesis), extraction, and/or purification of nicotine. It should be understood that the steps from various examples can be combined and/or performed in any suitable combination or order. For example, one or more steps from one example can be implemented with, or substituted for, one or more steps from another example, as desired and/or appropriate.

### Example 19

In various examples, nicotine can be formed via biosynthesis using a cell factory model. Creating a cell factory model can involve a controlled biotechnological system used to produce nicotine. A controlled biotechnological system can involve engineered microorganisms (e.g., E. coli, yeast (saccharomyces cerevisiae)).

A host microorganism can be selected for the biosynthesis. For example, E. coli can be selected because of its fast growth/reproduction, ease of genetic modification, high yield of heterologous proteins; S. cerevisiae can be selected because of its efficient alkaloid pathway expression.

The biosynthesis of nicotine can include an ornithine pathway for pyrrolidine portion formation, and/or a pyridine portion pathway for nicotinic acid production. A final condensation step can produce nicotine.

The following enzymes can be introduced and/or expressed: ODC (can convert L-ornithine to putrescine); PMT (can methylate putrescine to produce N-methylputrescine); N-methylputrescine oxidase (MPO) (can convert N-methylputrescine to N-methylpyrrolinium cation); quinolinate synthase (QS) (can synthesize quinolinic acid, a precursor for nicotinic acid); and/or nicotinic acid condensation enzyme (can catalyze the final reaction to form nicotine).

Various genetic engineering processes and/or steps can occur in the biosynthesis of nicotine. For example, gene cloning can occur, in which genes encoding enzymes from the tobacco plant (Nicotiana tabacum) are identified and cloned into the microbial host genome. Regarding promotors and regulators, strong inducible promoters to optimize enzyme expression can be used. To optimize the metabolic pathway, precursor availability can be balanced for the pyrrolidine portion and pyridine portion pathways using tools like CRISPR-Cas9 and/or synthetic biology platforms.

The precursor supply can be optimized to maximize nicotine production. For ornithine supply, pathways can be engineered in the host microorganism to increase ornithine availability (e.g., by overexpressing enzymes such as ornithine aminotransferase). For nicotinic acid, NAD salvage pathways/processes can be introduced and/or enhanced to facilitate a consistent or sufficient supply of nicotinic acid.

A fermentation process can be designed to support nicotine biosynthesis. For example, a nutrient-rich medium can be provided (e.g., to supplement precursors like ornithine and nicotinic acid). Specific inducers can be used (e.g., IPTG in E. coli or galactose in yeast) to activate engineered pathways. Environment conditions can be created and/or maintained to enhance secondary metabolite production (e.g., oxidative stress).

Producing nicotine can comprise pyrrolidine portion formation, which includes ODC decarboxylating L-ornithine to putrescine; PMT N-methylating putrescine to N-methylputrescine (using SAM as methyl donor); and/or (MPO) oxidatively deaminating N-methylputrescine to 4-(methylamino)butanal, which can (spontaneously) cyclize to the reactive N-methyl-Δ¹-pyrrolinium cation.

Producing nicotine can comprise pyridine portion formation, including QS condensing iminoaspartate (from L-aspartate) with dihydroxyacetone phosphate to quinolinic acid, and/or downstream native or enhanced enzymes (e.g., QPRT-like) can convert quinolinic acid to nicotinic acid (or activated derivative). Condensation to nicotine can comprise cryptic glucosylation (UGT-like) activating nicotinic acid, A622 (PIP oxidoreductase) reducing the activated intermediate, BBL enzyme (berberine bridge enzyme-like) oxidizing the coupled product, de-glucosylation (β-glucosidase) yielding (S)-nicotine via stereoselective Mannich-like condensation with N-methylpyrrolinium.

The product comprising nicotine can be extracted using an organic solvent (e.g., ethyl acetate or dichloromethane), two-phase liquid-liquid extraction, and/or adsorbent resins tailored for nicotine separation.

Formation of nicotine via a cell factory approach can have various benefits including sustainability (avoids reliance on large-scale tobacco cultivation, which requires significant land and resources), control (allows precise control over production conditions, reducing variability), scalability (engineered microbes can be cultivated at an industrial scale), and/or customization (enables production of nicotine derivatives or analogs for pharmaceutical applications). This process offers a sustainable, scalable pathway to produce nicotine for pharmaceutical, industrial, and/or research applications.

Of note, this example is related to and/or can implement any of the aspects of Example 16 regarding production of 6-methylnicotine discussed herein, or vice versa.

### Example 20

In various examples, biosynthesis of nicotine can occur via catalytic hydrogenation and/or regioselective alkylation.

A starting material can comprise pyridine and/or a pyridine derivative (e.g., a compound comprising a pyridine ring) (e.g., derived from nicotinic acid and/or synthesized de novo). In the catalytic hydrogenation of a pyridine derivative, a pyridine ring (e.g., derived from nicotinic acid or its derivatives) can undergo partial catalytic hydrogenation to introduce the desired saturation and prepare the pyridine ring for regioselective alkylation. A catalyst can be used, for example, comprising palladium on carbon (Pd/C) and/or Raney nickel. The reaction conditions can comprise mild pressure (H2 gas), a solvent including methanol, ethanol, and/or tetrahydrofuran (THF), and/or controlling the degree of hydrogenation to prevent full reduction of the aromatic ring. An example reaction can comprise:

C₅H₅N—H₂, Pd/C→Partially hydrogenated pyridine derivative.

Another starting material can comprise a pyrrolidine portion, which can be synthesized from precursors such as putrescine or similar amines. The pyrrolidine portion can be prepared via reductive amination and/or cyclization of a polyamine like putrescine. For example, putrescine can be reacted with an aldehyde or ketone to form a Schiff base, which can be reduced to a pyrrolidine portion. An example reaction can comprise:

H₂N—(CH₂)₄—NH₂+R—CHO—(H₂, Catalyst)→Pyrrolidine derivative (e.g., 1-methylpyrrolidine, N-methyl-Δ¹-pyrrolinium cation, (S)-nicotine (3-[(2S)-1-methylpyrrolidin-2-yl]pyridine), proline (pyrrolidine-2-carboxylic acid), and/or nornicotine (3-(pyrrolidin-2-yl)pyridine)).

The pyridine portion (e.g., a partially-hydrogenated pyridine ring) can be alkylated at the desired position (e.g., the 3-position) using a pyrrolidine portion. Reagents used in such a reaction can comprise base catalysts (e.g., NaH, K₂CO₃) and/or Lewis acids (e.g., BF₃, AlCl₃) to control regioselectivity. The reaction can proceed via a SN2 or SNAr mechanism, depending on substituents and conditions. A solvent can comprise aprotic solvents such as DMF and/or DMSO. Temperate of the reaction can be moderate to high (50-150° C.). An example reaction can comprise:

Partially hydrogenated pyridine+Pyrrolidine derivative—Catalyst→Nicotine

The product comprising nicotine can be purified by distillation and/or chromatography (e.g., to remove byproducts or regioisomers).

Formation of nicotine via catalytic hydrogenation and/or regioselective alkylation can have various benefits including controlled synthesis (enables regioselective introduction of functional groups for precision synthesis), scalability (can be suitable for both lab-scale and industrial-scale synthesis), and/or versatility (modifications in reagents or conditions can allow synthesis of nicotine analogs).

Of note, this example is related to the aspects of Example 1 regarding production of 6-methylnicotine discussed herein, and any such aspects can be applied and/or implemented to this example, or vice versa.

### Example 21

In various examples, biosynthesis of nicotine can occur via electrophilic aromatic substitution (EAS) and/or asymmetric catalysis. This approach can leverage reactivity of aromatic compounds and the stereochemical control facilitated by asymmetric catalysis to construct the pyridine and pyrrolidine portions of nicotine in a regio- and stereoselective manner. EAS can be used to functionalize the pyridine ring to enable subsequent reactions, such as alkylation or coupling with pyrrolidine derivatives. Asymmetric catalytic reactions can be employed to introduce stereochemical control during the coupling of the pyridine and pyrrolidine portions.

The pyridine ring can undergo selective substitution at a specific position(s). The reagents for a reaction can include an electrophile (e.g., acyl halides (RCOCl), alkyl halides (R—X), and/or nitro groups (NO₂) and/or a Lewis acid catalyst (e.g., AlCl₃, BF₃, or FeCl₃). A pyridine derivative can be reacted with an electrophile under controlled conditions to introduce functional groups, such as alkyl or acyl groups, in a regioselective manner. An example reaction can comprise:

C₅H₅N+RCOCl—AlCl₃→Functionalized pyridine derivative; and/or

Pyridine+NO₂⁺ (from HNO₃/H₂SO₄)→3-nitropyridine (major, meta-directed by protonated N).

A pyrrolidine portion can be synthesized via asymmetric hydrogenation or reductive amination of chiral intermediates. Asymmetric catalysts can comprise chiral transition-metal catalysts (e.g., Rh, Ru, or Ir complexes with ligands such as BINAP or PHOX), and/or organocatalysts. An example reaction can comprise: Imine intermediate+H₂→Chiral Rh catalyst→pyrrolidine derivative (e.g., 1-methylpyrrolidine, unsubstituted pyrrolidine, an/dor N-methyl-Δ¹-pyrrolinium cation).

The functionalized pyridine and pyrrolidine portions can be coupled to form the nicotine structure. Reagents for the reaction can comprise a strong base (e.g., NaH and/or K₂CO₃) and/or a catalyst to facilitate regioselective alkylation. For asymmetric catalysis, a chiral catalyst can be used to maintain stereochemical integrity during the coupling step. The reaction can proceed via an SN2 or nucleophilic substitution mechanism. An example reaction can comprise: Functionalized pyridine+pyrrolidine derivative→Base or catalyst→Nicotine.

The product comprising nicotine can be purified, for example via chromatography (separates regio- or stereoisomers) and/or distillation. The product can be characterized via NMR spectroscopy and/or chiral HPLC, for example, to confirm the structure and enantiopurity.

Formation of nicotine via EAS and asymmetric catalysis can have various benefits including regioselectivity (EAS can facilitate control over the substitution pattern on the pyridine ring), stereoselectivity (asymmetric catalysis can yield desired stereochemistry), versatility (can be adapted to synthesize nicotine analogs with specific structural modifications), and/or scalability (suitable for industrial-scale production of nicotine and its derivatives).

Of note, this example is related to the aspects of Example 2 regarding production of 6-methylnicotine discussed herein, and any such aspects can be applied and/or implemented to this example, or vice versa.

### Example 22

In various examples, biosynthesis of nicotine can occur via radical-based synthesis, which can involve employing radical chemistry to construct the pyridine and pyrrolidine portions and their regioselective coupling.

Radical-based synthesis of nicotine can comprise functionalizing a pyridine portion, forming or providing a pyrrolidine portion, and/or coupling the pyridine and pyrrolidine portions selectively to form nicotine. Radical intermediates can be generated using, e.g., photoredox catalysis (light-driven radical formation), thermal and/or chemical initiation (e.g., azo compounds or peroxides), and/or electrochemical methods for precise radical control.

A pyridine ring can be functionalized to create a reactive intermediate for coupling. Alkyl halides (R—X) can be used as radical precursors. Radicals can be generated via photoredox catalysis (e.g., Ir or Ru complexes) and/or chemical initiators (e.g., azobisisobutyronitrile, AIBN). In the reaction, the radical can react with the electron-deficient pyridine ring, selectively adding to a specific position. Reagents can include alkyl iodides (e.g., methyl iodide) and photoredox catalysts under visible light. An example reaction can comprise: C₅H₅N+R—X→Catalyst, Light→Alkyl-pyridine derivative; and/or C5H5N+HCO2H (formic acid as H-donor)—[Cp*RhCl2]2 catalyst, RT, MeCN/H2O solvent→1,2,3,6-tetrahydropyridine.

To form a pyrrolidine portion, a starting compound can comprise an unsaturated amine and/or a dihalide precursor. Radicals can be generated at one end of the molecule (e.g., via photoredox or electrochemical methods). The radical can undergo intramolecular cyclization to form a five-membered pyrrolidine ring. An example reaction (e.g., with starting materials comprising N-substituted aminoalkenes or dihaloalkanes) can comprise: H₂N—(CH₂)₄—X→Radical Initiator→Pyrrolidine derivative.

The functionalized pyridine and pyrrolidine portions can be coupled to form nicotine. Radicals can be generated on the pyridine and/or pyrrolidine portion. The radicals can combine regioselectively to form the C—C bond. Catalysts for the reaction can comprise transition metals such as Ni or Cu, or compounds including the same. Any remaining functional groups can be modified to yield the final nicotine structure. For example, N-methylation of the pyrrolidine ring can be achieved using a reagent such methyl iodide (CH₃I) and/or dimethyl sulfate.

Formation of nicotine via radical-based synthesis can have various benefits including mild conditions (radicals can be generated under photochemical or electrochemical conditions, avoiding harsh reagents or temperatures), high efficiency (radical reactions can proceed rapidly and efficiently, enabling faster synthesis), regioselectivity (radical intermediates can be directed to specific positions on the pyridine and pyrrolidine portion), versatility (compatible with a wide range of starting materials and functional groups), and/or scalability (radicals can be easily generated in continuous flow systems, making this approach scalable). Radical-based synthesis can be advantageous for synthesizing complex molecules with challenging substitution patterns, such as nicotine, and is suitable for both pharmaceutical and fine chemical industries.

Of note, this example is related to the aspects of Example 3 regarding production of 6-methylnicotine discussed herein, and any such aspects can be applied and/or implemented to this example, or vice versa.

### Example 23

In various examples, biosynthesis of nicotine can occur via biomimetic synthesis. This approach utilizes simple precursors and can resemble enzyme-catalyzed reactions in plants.

In plants, nicotine is synthesized by pyrrolidine ring formation (e.g., from ornithine and/or arginine, which leads to N-methylpyrrolinium cation); pyridine ring formation (e.g., from the pyridine nucleotide cycle, producing nicotinic acid), and/or a coupling step (the N-methylpyrrolinium cation reacts with a nicotinic acid derivative to form nicotine).

A starting material can comprise putrescine and/or a similar polyamine. Putrescine can be converted into a pyrrolidine portion via a decarboxylation or cyclization reaction. The pyrrolidine portion can be methylated using methyl iodide (CH3I) or dimethyl sulfate to form N-methylpyrrolinium iodide. An example reaction can comprise:

Putrescine→Cyclization→Pyrrolidine→CH₃I→N-methylpyrrolinium cation.

Another starting material can comprise nicotinic acid and/or 3-cyanopyridine. The acid or nitrile group can be reduced to form a reactive intermediate for coupling with a pyrrolidine portion. An example reaction can comprise: Nicotinic acid→Reduction→Pyridine intermediate.

In various examples, for the pyrrolidine portion, putrescine can form N-methylputrescine (e.g., via reductive methylation: HCHO/HCO₂H or HCHO/NaBH₃CN). Oxidation (biomimetic MPO: Cu-amine oxidase mimic or H₂O₂)→4-(methylamino)butanal→(spontaneous) cyclization in mild buffer (pH 5-8, RT)→N-methyl-Δ¹-pyrrolinium cation. For the pyridine portion, nicotinic acid→activation (e.g., to nicotinoyl thioester mimic or dihydronicotinic acid via partial reduction). A 1,4-dihydronicotine intermediate (Na₂S₂O₄ or enzymatic mimic) can be reduced for nucleophilic reactivity.

To couple the pyrrolidine and pyridine portions, base-catalyzed alkylation and/or transition metal catalysts can be used to form the C—C bond between the pyridine and pyrrolidine portions. A nucleophilic pyridine intermediate and/or electrophilic N-methylpyrrolinium cation can be used for the coupling reaction. An example reaction can comprise: N-Methylpyrrolinium cation+Pyridine intermediate→Base or Catalyst→Nicotine.

Reaction conditions can be mild (e.g., mild temperatures (at or around room temperature)), and/or environmentally benign solvents (e.g., water or ethanol). Biomimetic catalysts, such as organocatalysts and/or metal complexes, can be employed to simulate enzymatic activity. Chiral catalysts and/or reagents can be sued to replicate the stereochemical fidelity of natural biosynthesis.

Formation of nicotine via biomimetic synthesis can have various benefits including scalability (enables large-scale production using simple and inexpensive starting materials), environmental friendliness (uses fewer harsh chemicals and milder conditions compared to traditional synthetic methods), and/or versatility (can be adapted to produce nicotine analogs by modifying the pyridine or pyrrolidine precursors).

Of note, this example is related to the aspects of Example 4 regarding production of 6-methylnicotine discussed herein, and any such aspects can be applied and/or implemented to this example, or vice versa.

### Example 24

In various examples, biosynthesis of nicotine can occur via template-directed synthesis. Template-directed synthesis for nicotine biosynthesis can involve templates that guide the assembly of molecular components with high precision and selectivity. In the case of nicotine, template-directed synthesis can be applied to guide the formation and coupling of the pyridine and pyrrolidine portions.

A molecular template can act as a scaffold to orient precursors correctly for the coupling reaction, facilitate bond formation through proximity effects, and/or enhance regio- and stereoselectivity. Templates can be organic scaffolds (macromolecules or small ligands with functional groups that direct reactants), metal complexes (metal centers that coordinate with precursors and guide their reactivity), and/or biomolecular mimics (peptide and/or nucleotide-like templates that replicate enzyme binding sites).

A pyridine precursor can be prepared by starting with nicotinic acid and/or 3-cyanopyridine, and/or modifying the precursor to comprise reactive sites (e.g., halogens or carbonyl groups) for coupling. A pyrrolidine precursor can be prepared by synthesizing or isolating N-methylpyrrolinium cation via methylation of pyrrolidine. An example reaction can comprise: Pyrrolidine+CH3I→N-Methylpyrrolinium cation.

A template can be designed with binding sites for both the pyridine and/or pyrrolidine precursors. Possible examples include macrocyclic templates (structures like crown ethers or cyclodextrins to hold the precursors), metal complexes (use metals (e.g., Zn²⁺ or Cu²⁺) that coordinate with both precursors), and/or peptidic scaffolds (short peptide chains with binding pockets).

Pyridine and/or pyrrolidine portions can bind to specific sites on the template via, e.g., hydrogen bonding, metal coordination, π-π interactions, and/or Van der Waals forces. The template can position the precursors such that their reactive groups align optimally for coupling.

The proximity of the pyridine and pyrrolidine portions can facilitate the coupling reaction. Bond formation can occur under mild conditions (e.g., low temperature, catalytic base). A catalyst (e.g., transition metals or organocatalysts) can assist the reaction. An example reaction can comprise: N-Methylpyrrolinium cation+Pyridine intermediate→Template→Nicotine.

After coupling the pyridine and pyrrolidine portions, nicotine can be released from the template by solvent extraction, mild heating, and/or pH adjustment. Resulting nicotine can be purified via chromatography and/or distillation.

Formation of nicotine via template-directed synthesis can have various benefits including enhanced selectivity (the template ensures high regio- and stereoselectivity by aligning precursors precisely), efficiency (proximity effects reduce the activation energy for bond formation), adaptability (templates can be customized to synthesize nicotine analogs), and/or mild conditions (reduces use of harsh chemicals or extreme reaction conditions). Template-directed synthesis can be particularly useful for small-scale, high-purity applications such as pharmaceutical research or the production of complex natural product derivatives.

Of note, this example is related to the aspects of Example 5 regarding production of 6-methylnicotine discussed herein, and any such aspects can be applied and/or implemented to this example, or vice versa.

### Example 25

In various examples, biosynthesis of nicotine can occur via flow chemistry synthesis, which integrates continuous reaction systems to synthesize nicotine. In the context of nicotine biosynthesis, the flow chemistry approach can enable continuous, controlled reactions. The advantages include improved safety, better reaction control, and scalability.

In flow chemistry synthesis, reaction can occur in a continuous stream rather than in batches, allowing precise control over reaction parameters such as temperature, pressure, and reaction time. Use of microreactors or tubular reactors with small channel dimensions can enhance mixing and heat transfer. Multiple steps, including precursor synthesis, coupling reactions, and purification, can be integrated into a single automated setup.

The flow synthesis process can be divided into steps including: pyrrolidine portion formation, pyridine portion preparation, and/or coupling the pyrrolidine and pyridine portions to form nicotine.

Cyclization of putrescine and/or reductive amination of aminoalkenes can be used to form the pyrrolidine portion. To complete this reaction, a solution of putrescine (or a similar amine precursor) can be passed through a flow reactor with a suitable catalyst (e.g., Pd/C and/or a homogeneous catalyst). Reductive conditions are maintained using hydrogen gas or a reducing agent (e.g., sodium borohydride). Pyrrolidine can be continuously collected in the output stream. An example reaction can comprise: Putrescine+H₂→Pd/C in flow→Pyrrolidine. The pyrrolidine stream can be combined with methylating agents (e.g., methyl iodide or dimethyl sulfate) in a second flow step to produce N-methylpyrrolinium iodide.

To prepare a pyridine portion, nicotinic acid or 3-cyanopyridine can be reduced to form a reactive intermediate (e.g., dihydropyridine derivatives). For example, nicotinic acid can be introduced into a flow reactor with reducing agents (e.g., lithium aluminum hydride) under controlled conditions. A catalyst can be used for selective reduction, such as Ru-based complexes. The output can be a continuous stream of the reduced pyridine intermediate. The pyridine intermediate can be alkylated or functionalized in a subsequent flow step to prepare it for coupling.

To couple the pyrrolidine and pyridine portions, the pyrrolidine and pyridine portions can be combined in a flow reactor for regioselective coupling to form nicotine. Coupling agents can comprise base-catalyzed coupling (e.g., NaH and/or K₂CO₃ in solution), and/or transition metal-catalyzed coupling (e.g., Ni, Cu, and/or Pd complexes). For the flow setup, two streams containing the precursors can be mixed at a T-junction and passed through a heated tubular reactor. Reaction conditions (temperature, pressure, residence time) can be optimized to favor coupling. An example reaction can comprise: N-Methylpyrrolinium iodide+Pyridine intermediate→Base/Catalyst in flow→Nicotine. The coupled product can be (continuously) collected as output, minimizing exposure to reactive intermediates.

Purification of the product comprising nicotine can occur inline, e.g., using liquid-liquid extraction (organic solvents to separate nicotine), and/or membrane separation (removal of byproducts). Inline purification can facilitate product quality and reduce downstream processing. The nicotine can be isolated as a pure product, ready for use and/or further derivatization.

Formation of nicotine via flow chemistry synthesis can have various benefits including improved safety (reduces and/or minimizes handling of toxic intermediates like N-methylpyrrolinium iodide or reactive pyridine derivatives), enhanced control (precise regulation of temperature, pressure, and residence time leads to higher yields and fewer byproducts), scalability (continuous flow systems can be easily scaled up by increasing the reactor size or running multiple reactors in parallel), efficiency (shorter reaction times due to enhanced mixing and heat transfer in microreactors, and/or integration of multiple steps into a single setup reduces overall processing time), and/or environment benefits (reduced or minimized solvent use and waste generation, making the process more sustainable).

Of note, this example is related to the aspects of Example 6 regarding production of 6-methylnicotine discussed herein, and any such aspects can be applied and/or implemented to this example, or vice versa.

### Example 26

In various examples, biosynthesis of nicotine can occur via solid-phase synthesis, which utilizes immobilized intermediates on a resin to facilitate sequential synthesis steps with high stereochemical control (e.g., greater than 80% or 90% enantiomeric excess) and ease of purification. In this method, intermediates can be covalently attached to a solid support (resin) and undergo sequential chemical transformations. After the synthesis is complete, the final product can be cleaved from the resin.

A resin and/or polymer bead with reactive functional groups (e.g., hydroxyl, amine, or carboxyl) can anchor one of the precursors (e.g., pyridine and/or pyrrolidine precursors or compounds). A common support can comprise polystyrene resin, PEG-based resins, and/or other cross-linked polymer beads. Nicotine's pyridine and pyrrolidine portions can be assembled stepwise, with intermediates attached to the solid support at each step. The initial precursor (e.g., pyridine and/or pyrrolidine precursors or portions) can be covalently attached to the resin. The produced nicotine can be released from the solid support in the final step via cleavage reactions.

Beginning with a pyridine portion, the pyridine portion can be functionalized (e.g., via carboxylation or halogenation) to create a reactive group for covalent attachment to the resin. The functionalized pyridine portion can be attached to the resin using coupling agents such as DCC (dicyclohexylcarbodiimide) and/or EDC (1-ethyl-3-(3-dimethylaminopropyl)carbodiimide). An example reaction can comprise: Pyridine derivative+Resin→Coupling agent→Pyridine-resin complex.

The pyrrolidine portion can be added. For example, a functionalized pyrrolidine portion (e.g., N-methylpyrrolinium salt) can be introduced and reacts with the immobilized pyridine. A base or catalyst can be used to facilitate nucleophilic substitution or reductive amination between the two fragments. If the pyrrolidine portion is not methylated, a methyl group can be added at this stage using a reagent like methyl iodide (CH₃I) or dimethyl sulfate. An example reaction can comprise: Pyridine-resin complex+Pyrrolidine precursor→Catalyst→Resin-bound nicotine intermediate.

For the coupling reaction to form nicotine, the immobilized pyridine and pyrrolidine components can undergo a coupling reaction to form the C—C bond linking the two rings. The coupling reaction, can be catalyzed by a base (e.g., NaH, K2CO3), and/or transition metals (e.g., Ni, Cu, and/or Pd complexes). An example reaction can comprise: Pyridine-resin complex+Pyrrolidine precursor→Base/Catalyst→Resin-bound nicotine.

The final nicotine product can be cleaved from the resin using a suitable cleavage reagent. For example, a cleavage strategy can comprise acidic cleavage (e.g., using trifluoroacetic acid, TFA), and/or reductive cleavage (e.g., using hydrogen and Pd/C). Cleavage may be conducted to minimize or prevent damaging the nicotine molecule. An example reaction can comprise: Resin-bound nicotine→Cleavage agent→Free nicotine.

The nicotine can be purified using liquid-liquid extraction and/or chromatography to remove residual resin and byproducts.

Formation of nicotine via solid-phase synthesis can have various benefits including ease of purification (intermediates remain attached to the solid support, simplifying purification via washing steps), automation (solid-phase synthesis is compatible with automated synthesis systems, enhancing efficiency and scalability), high yield (minimize loss of intermediates due to immobilization on the resin), and/or scalability (can be scaled up by increasing the quantity of resin used). By immobilizing intermediates, solid-phase synthesis simplifies purification and enhances reaction efficiency, making this process a valuable tool in synthetic organic chemistry.

Of note, this example is related to the aspects of Example 7 regarding production of 6-methylnicotine discussed herein, and any such aspects can be applied and/or implemented to this example, or vice versa.

### Example 27

In various examples, biosynthesis of nicotine can occur via microbial engineering, which uses genetically modified microorganisms to biosynthesize nicotine by integrating tailored enzymes and metabolic networks.

For this process, a microbial host can be selected, such as E. coli (for its rapid growth well-studied genetics, and high productivity), yeast (S. cerevisiae) (which can support more complex metabolic pathways and post-translational modifications), and/or cyanobacteria (which uses sunlight and carbon dioxide as resources and reducing feedstock costs). A host organism can be selected for various reasons, such as ease of genetic manipulation and/or tolerance to nicotine (to avoid toxicity during production).

Precursors can comprise ornithine and/or arginine (for the pyrrolidine portion) and/or nicotinic acid (e.g., from the NAD degradation/salvage pathway) (for the pyridine portion). These precursors can be supplied externally in the culture medium and/or produced directly by the host organism through metabolic engineering.

Biosynthesis of nicotine can utilize enzymes including ODC (can convert ornithine into putrescine), PMT (can methylate putrescine to form N-methylputrescine), MPO (can convert N-methylputrescine into N-methylpyrrolinium cation), QS (can catalyze the production of nicotinic acid from NAD), and/or nicotine synthase (NS) (and/or similar enzymes) (can catalyze the condensation of the N-methylpyrrolinium cation with nicotinic acid or related intermediates to form nicotine). In various examples, the selected host organism may only express certain enzymes, e.g., only PMT, MPO, and/or NS. The precursors can be supplied externally.

In various examples, genetic engineering tools such as CRISPR-Cas9 or plasmid-based expression can be used to overexpress certain enzymes (e.g., PMT, MPO, and/or NS), and/or knock out competing metabolic pathways that consume precursors. Pathway flux can be balanced by regulating enzyme expression levels to maximize nicotine production.

To ferment and accumulate nicotine, the engineered microorganisms can be grown in a bioreactor with optimized conditions, such as a temperature of 30-37° C. (which may be optimal for microbial growth), a pH of 6.5-7.0, and/or a nutrient-rich medium, supplemented with ornithine or nicotinic acid if needed. Nicotine can be secreted into the medium or retained in the cell, depending on the microbial system.

The fermentation broth can be harvested, and the nicotine comprised therein can be harvested using liquid-liquid extraction (e.g., using organic solvents (e.g., chloroform, ethyl acetate) to separate nicotine), and/or adsorption chromatography (for further purification). High-purity nicotine may be yielded suitable for research or industrial applications.

Formation of nicotine via microbial engineering can have various benefits including ease of implementation (utilizes established genetic engineering techniques and commercially available microorganisms), scalability (fermentation-based approach is easily scalable for industrial production), cost-effectiveness (produces nicotine directly without complex chemical steps and/or plant cultivation), environmental sustainability (avoids tobacco farming and associated environmental concerns), and/or customizability (enables production of nicotine analogs by modifying the biosynthetic pathways). This microbial fermentation process can be suitable for large-scale production where sustainability, cost-efficiency, and high stereochemical purity are important.

Of note, this example is related to the aspects of Example 9 regarding production of 6-methylnicotine discussed herein, and any such aspects can be applied and/or implemented to this example, or vice versa.

The processes and steps in these examples of nicotine preparation can be used to form other nicotine derivatives (e.g., 6-methylnicotine), for example by the precursors comprising (or being used to form) pyridine and pyrrolidine portions/moieties that, when couple, form 6-methylnicotine or other desired nicotine derivatives. Thus, the disclosure of these examples can apply to the synthesis of 6-methylnicotine.

In the detailed description, references to “various examples”, “one example”, “an example”, etc., indicate that the examples described may include a particular feature, structure, process step, reaction, characteristic, or other aspect, but every example may not necessarily include the particular feature, structure, process step, reaction, characteristic, or other aspect. Moreover, such phrases are not necessarily referring to the same example. Further, when a particular feature, structure, process step, reaction, characteristic, or other aspect is described in connection with an example, it is submitted that it is within the knowledge of one skilled in the art to effect or implement such feature, structure, process step, reaction, characteristic, or other aspect in connection with other examples whether or not explicitly described. After reading the description, it will be apparent to one skilled in the relevant art(s) how to implement the disclosure in alternative embodiments.

Steps recited in any of the method or process descriptions may be executed in any order or combination and are not necessarily limited to the order presented. The steps from different processes or examples can be combined between processes/examples in any suitable manner, and are not limited to the specific combination and process of any one specific example. Furthermore, any reference to singular includes plural examples, and any reference to more than one component or step may include a singular example or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

As used herein, the term “and/or” includes any and all combinations of one or more of the associated listed items. Unless otherwise noted, expressions such as “at least one of,” when preceding a list of elements, modify the entire list of elements and do not necessarily modify the individual elements of the list.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A method, comprising:
providing a pyridine compound;
providing a pyrrolidine compound; and
coupling the pyridine compound and the pyrrolidine compound to form a nicotine compound.

2. The method of claim 1, wherein the pyrrolidine compound comprises N-methylpyrrolidine.

3. The method of claim 2, wherein the pyridine compound comprises 2-methylpyridine.

4. The method of claim 3, wherein the nicotine compound comprises 6-methylnicotine.

5. The method of claim 4, wherein the coupling the pyridine compound and the pyrrolidine compound comprises stereocontrolled coupling, preferentially forming S-6-methylnicotine.

6. The method of claim 2, wherein the pyridine compound comprises pyridine.

7. The method of claim 6, wherein the nicotine compound comprises nicotine.

8. The method of claim 1, wherein the providing the pyridine compound comprises forming the pyridine compound from a pyridine precursor.

9. The method of claim 1, wherein the providing the pyrrolidine compound comprises forming the pyrrolidine compound from a pyrrolidine precursor.

10. The method of claim 9, wherein the pyrrolidine precursor comprises a linear amine.

11. The method of claim 10, wherein the linear amine comprises at least one of putrescine or ornithine.

12. The method of claim 1, wherein the pyridine compound comprises a pyridine ring, and wherein the method further comprises:
reducing the pyridine compound using a palladium catalyst; and
alkylating the pyridine ring.

13. The method of claim 12, further comprising activating the pyridine compound via halogenation before the coupling the pyridine compound and the pyrrolidine compound.

14. The method of claim 13, wherein the activating the pyridine compound via halogenation comprises reacting the pyridine compound with at least one of N-bromosuccinimide or a compound comprising iodine; and wherein the coupling the pyridine compound and the pyrrolidine compound comprises reacting the pyridine compound and the pyrrolidine compound under basic conditions,
wherein the nicotine compound comprises 6-methylnicotine.

15. The method of claim 14, further comprising:
extracting the nicotine compound; and
purifying the nicotine compound, wherein the purified nicotine compound is devoid of nicotine.

16. The method of claim 9, wherein the providing the pyridine compound comprises:
mixing a methylating agent with the pyridine compound in the presence of a catalyst to methylate the pyridine compound; and
halogenating the pyridine compound at the 5-position; and
wherein the providing the pyrrolidine compound comprises:
mixing at least one of an aldehyde reagent or a ketone reagent with the pyrrolidine precursor to cyclize the pyrrolidine precursor.

17. The method of claim 16, wherein the providing the pyridine compound, the providing the pyrrolidine compound, and the coupling the pyridine compound and the pyrrolidine compound to form a nicotine compound occur in a single reaction vessel.

18. The method of claim 11, further comprising utilizing a microbial host,
wherein the providing the pyridine compound comprises:
forming nicotinic acid from quinolinic acid via quinolinate phosphoribosyltransferase;
methylating the nicotinic acid at the 6-position of the nicotinic acid via 6-methyltransferase, forming 6-methylnicotinic acid; and
decarboxylating the 6-methylnicotinic acid; and
wherein the providing the pyrrolidine compound comprises:
forming putrescine from ornithine via ornithine decarboxylase; and
cyclizing the putrescine into the pyrrolidine compound.

19. The method of claim 11, wherein the linear amine comprises ornithine,
wherein the providing the pyridine compound comprises:
condensing iminoaspartate with dihydroxyacetone phosphate to quinolinic acid via quinolinate synthase; and
converting the quinolinic acid to nicotinic acid;
wherein the providing the pyrrolidine compound comprises:
decarboxylating the ornithine to putrescine via ornithin decarboxylase;
N-methylating the putrescine to N-methylputrescine via putrescine N-methyltransferase;
deaminating the N-methylputrescine to 4-(methylamino)butanal via N-methylputrescine oxidase; and
cyclizing the 4-(methylamino)butanal to form N-methylpyrrolinium.

20. The method of claim 19, wherein the coupling the pyridine compound and the pyrrolidine compound to form a nicotine compound comprises:
activating the nicotinic acid via cryptic glucosylation, forming an activated nicotinic acid compound;
reducing the activated nicotinic acid compound via oxidoreductase, forming a reduced nicotinic acid compound;
coupling the reduced nicotinic acid compound with the N-methylpyrrolinium; and
de-glucosylating the nicotine compound,
wherein the nicotine compound comprises nicotine.
